(19) United States
(12) Patent Application Publication
(10) Pub. No.: US 2024/0114781 A1
(43) Pub. Date: Apr. 4, 2024

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Masaki MORITA, Kanagawa (JP); Ryo FUJIWARA, Kanagawa (JP); Hiroki SUGIURA, Kanagawa (JP); Yasunori YONEKUTA, Kanagawa (JP); Hiyoku NAKATA, Kanagawa (JP)

(73) Assignee: FUJIFILM Corporation, Tokyo (JP)

(21) Appl. No.: 18/307,789

(22) Filed: Apr. 26, 2023

Related U.S. Application Data
(63) Continuation of application No. PCT/JP2021/038036, filed on Oct. 14, 2021.

(30) Foreign Application Priority Data
Oct. 30, 2020 (JP) 2020-183043

(51) Int. Cl. H10K 85/60; C07D 495/04; C07D 519/00; H10K 30/20

(52) U.S. Cl. CPC H10K 85/657; C07D 495/04; C07D 519/00; H10K 30/20; H10K 85/6572; H10K 85/6576

(57) ABSTRACT

The present invention is to provide a photoelectric conversion element with an excellent photoelectric conversion efficiency, an imaging element, an optical sensor, and a compound. The photoelectric conversion element of the present invention includes, in the following order, a conductive film, a photoelectric conversion film, and a transparent conductive film, in which the photoelectric conversion film contains a compound represented by Formula (1).

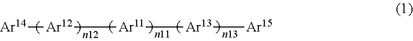

### CROSS-REFERENCE TO RELATED APPLICATIONS

This application is a Continuation of PCT International Application No. PCT/JP2021/038036 filed on Oct. 14, 2021, which claims priority under 35 U.S.C. § 119(a) to Japanese Patent Application No. 2020-183043 filed on Oct. 30, 2020. The above applications are hereby expressly incorporated by reference, in its entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, the development of an element (for example, an imaging element) having a photoelectric conversion film has been progressing.

For example, it is disclosed in JP2018-170487A that a photoelectric conversion element for an imaging element includes a photoelectric conversion unit having one or more layers of an organic thin film layer containing a predetermined compound.

### SUMMARY OF THE INVENTION

In recent years, along with the demand for improving the performance of imaging elements, optical sensors, and the like, further improvements are required for various characteristics required for photoelectric conversion elements used therein.

For example, further improvements are required for a photoelectric conversion efficiency in the photoelectric conversion elements.

The present inventors have studied the photoelectric conversion element formed of compounds disclosed in JP2018-170487A, and have confirmed that there is room for improving a photoelectric conversion efficiency (for example, a photoelectric conversion efficiency for light having a wavelength of 400 to 550 nm) in such a photoelectric conversion element.

In view of the above circumstances, an object of the present invention is to provide a photoelectric conversion element with an excellent photoelectric conversion efficiency.

Another object of the present invention is to provide an imaging element, an optical sensor, and a compound related to the above-described photoelectric conversion element.

The present inventors have conducted extensive studies on the above-described problems, and as a result, the inventors have found that it is possible to solve the above-described problems by configurations described below and have completed the present invention.

[1]

A photoelectric conversion element comprising, in the following order:
a conductive film;
a photoelectric conversion film; and
a transparent conductive film,
in which the photoelectric conversion film contains a compound represented by Formula (1),

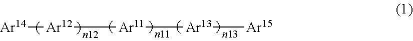

in Formula (1), n11 represents 1 or 2,
n12 and n13 each independently represent 0 or 1, where, at least one of n12 or n13 represents 1,
Ar¹¹ represents a fused polycyclic aromatic ring group consisting of a combination of one or more aromatic rings selected from the group consisting of a thiophene ring, a benzene ring, a furan ring, and a selenophene ring, where, the fused polycyclic aromatic ring group has three or four rings, the fused polycyclic aromatic ring group may have a substituent,
Ar¹⁴ and Ar¹¹ each independently represent an aryl group which may have a substituent or a heteroaryl group which may have a substituent, and
Ar¹² and Ar¹³ each independently represent any one of groups represented by Formula (2) to Formula (4),

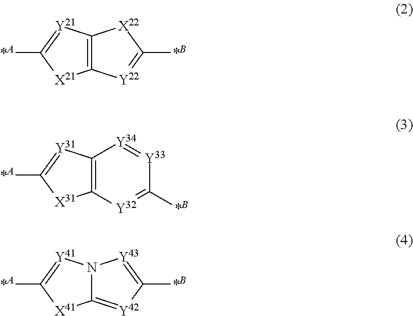

in Formula (2) to Formula (4), *^{A} and *^{B} each represent a bonding position,
in Formula (2), X²¹ and X²² each independently represent a sulfur atom, an oxygen atom, or a selenium atom, Y²¹ and Y²² each independently represent a nitrogen atom or —CR═, and R represents a hydrogen atom or a substituent, where, at least one of Y²¹ or Y²² represents a nitrogen atom,
in Formula (3), X³¹ represents a sulfur atom, an oxygen atom, or a selenium atom, Y³¹ to Y³⁴ each independently represent a nitrogen atom or —CR═, and R represents a hydrogen atom or a substituent,
in Formula (4), X⁴¹ represents a sulfur atom, an oxygen atom, or a selenium atom, Y⁴¹ to Y⁴³ each independently represent a nitrogen atom or —CR═, and R represents a hydrogen atom or a substituent.
   [2]

The photoelectric conversion element according to [1], in which in Formula (1), a group represented by Ar¹¹ is any one of groups represented by Formula (A1) to Formula (A6),

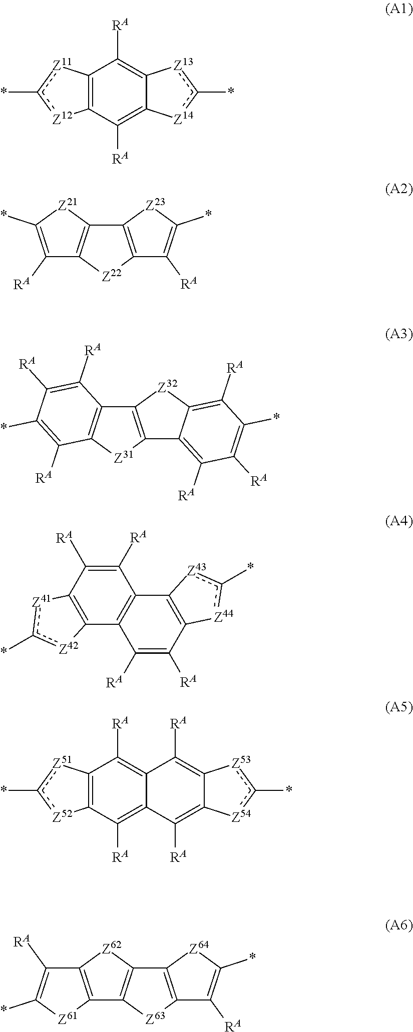

in Formula (A1), one of Z¹¹ or Z¹² represents a sulfur atom, an oxygen atom, or a
selenium atom, and the other of Z¹¹ or Z¹² represents —CR═, one of Z¹³ or Z¹⁴ represents a sulfur atom, an oxygen atom, or a selenium atom, and the other of Z¹³ or Z¹⁴ represents —CR═, and R and R^{A} each independently represent a hydrogen atom or a substituent,
in Formula (A2), Z²¹ to Z²³ each independently represent a sulfur atom, an oxygen atom, or a selenium atom, and R^{A} represents a hydrogen atom or a substituent,
in Formula (A3), Z³¹ and Z³² each independently represent a sulfur atom, an oxygen atom, or a selenium atom, and R^{A} represents a hydrogen atom or a substituent,
in Formula (A4), one of Z⁴¹ or Z⁴² represents a sulfur atom, an oxygen atom, or a selenium atom, and the other of Z⁴¹ or Z⁴² represents —CR═, one of Z⁴³ or Z⁴⁴ represents a sulfur atom, an oxygen atom, or a selenium atom, and the other of Z⁴³ or Z⁴⁴ represents —CR═, and R and R^{A} each independently represent a hydrogen atom or a substituent,
in Formula (A5), one of Z⁵¹ or Z⁵² represents a sulfur atom, an oxygen atom, or a selenium atom, and the other of Z⁵¹ or Z⁵² represents —CR═, one of Z⁵³ or Z⁵⁴ represents a sulfur atom, an oxygen atom, or a selenium atom, and the other of Z⁵³ or Z⁵⁴ represents —CR═, and R and R^{A} each independently represent a hydrogen atom or a substituent,
in Formula (A6), Z⁶¹ to Z⁶⁴ each independently represent a sulfur atom, an oxygen atom, or a selenium atom, and R^{A} represents a hydrogen atom or a substituent.
   [3]

The photoelectric conversion element according to [2], in which in Formula (A1), one of Z¹¹ or Z¹² represents a sulfur atom or an oxygen atom, and the other of Z¹¹ or Z¹² represents —CR═, and
one of Z¹³ or Z¹⁴ represents a sulfur atom or an oxygen atom, and the other of Z¹³ or Z¹⁴ represents —CR═,
in Formula (A2), Z²¹ to Z²³ each independently represent a sulfur atom or an oxygen atom,
in Formula (A3), Z³¹ and Z³² each independently represent a sulfur atom or an oxygen atom,
in Formula (A4), one of Z⁴¹ or Z⁴² represents a sulfur atom or an oxygen atom, and the other of Z⁴¹ or Z⁴² represents —CR═, and
one of Z⁴³ or Z⁴⁴ represents a sulfur atom or an oxygen atom, and the other of Z⁴³ or Z⁴ represents —CR═,
in Formula (A5), one of Z⁵¹ or Z⁵² represents a sulfur atom or an oxygen atom, and the other of Z⁵¹ or Z⁵² represents —CR═, and
one of Z⁵³ or Z⁵⁴ represents a sulfur atom or an oxygen atom, and the other of Z⁵³ or Z⁵⁴ represents —CR═, and
in Formula (A6), Z⁶¹ to Z⁶⁴ each independently represent a sulfur atom or an oxygen atom.
   [4]

The photoelectric conversion element according to [2] or [3], in which in Formula (1), n11 is 1, and Ar¹¹ is a group represented by Formula (A5).

[5]

The photoelectric conversion element according to any one of [1] to [4], in which in Formula (2), X²¹ and X²² each independently represent a sulfur atom or an oxygen atom,
in Formula (3), X³¹ represents a sulfur atom or an oxygen atom, and
in Formula (4), X⁴¹ represents a sulfur atom or an oxygen atom.
   [6]

The photoelectric conversion element according to any one of [1] to [5], in which in Formula (1), Ar¹² and Ar¹³ each independently represent any one of groups represented by Formula (5) to Formula (13),

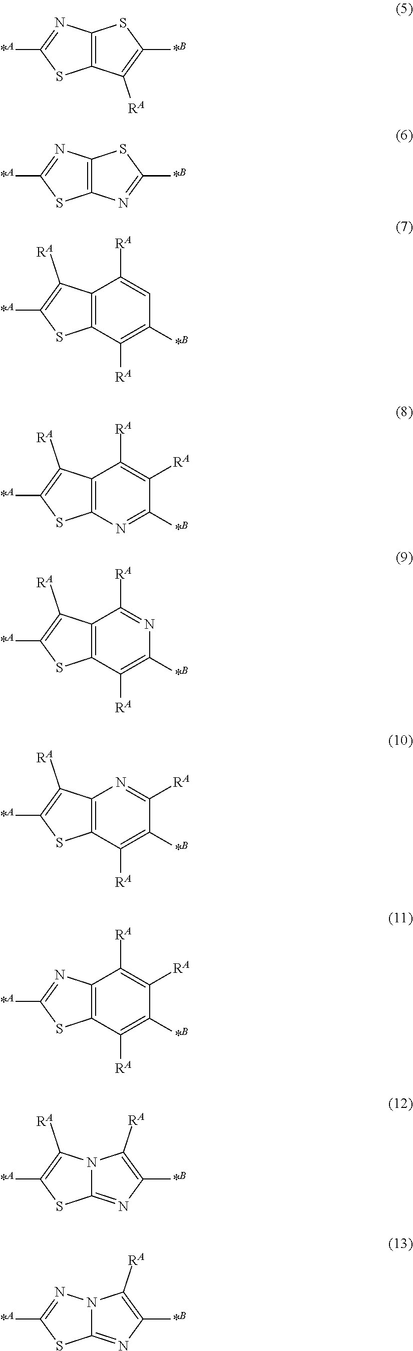

in Formula (5) to Formula (13), *^{A} and *^{B} each represent a bonding position, and
R^{A} represents a hydrogen atom or a substituent.
   [7]

The photoelectric conversion element according to any one of [1] to [6], in which the compound represented by Formula (1) has a molecular weight of 550 to 1200.

[8]

The photoelectric conversion element according to any one of [1] to [7], in which the photoelectric conversion film further contains a coloring agent, and
the photoelectric conversion film is a mixture layer formed in a state in which the compound represented by Formula (1) and the coloring agent are mixed.
   [9]

The photoelectric conversion element according to any one of [1] to [8], in which the photoelectric conversion film further contains a n-type semiconductor material.

[10]

The photoelectric conversion element according to [9], in which the n-type semiconductor material includes fullerenes selected from the group consisting of a fullerene and a derivative thereof.

[11]

The photoelectric conversion element according to any one of [1] to [10], further comprising one or more interlayers between the conductive film and the transparent conductive film, in addition to the photoelectric conversion film.

[12]

An imaging element comprising the photoelectric conversion element according to any one of [1] to [11].

[13]

An optical sensor comprising the photoelectric conversion element according to any one of [1] to [11].

[14]

A compound represented by Formula (1),

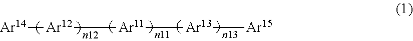

in Formula (1), n11 represents 1 or 2,
n12 and n13 each independently represent 0 or 1, where, at least one of n12 or n13 represents 1,
Ar¹¹ represents a fused polycyclic aromatic ring group consisting of a combination of one or more aromatic rings selected from the group consisting of a thiophene ring, a benzene ring, a furan ring, and a selenophene ring, where, the fused polycyclic aromatic ring group has three or four rings, the fused polycyclic aromatic ring group may have a substituent,
Ar¹⁴ and Ar¹⁵ each independently represent an aryl group which may have a substituent or a heteroaryl group which may have a substituent, and
Ar¹² and Ar¹³ each independently represent any one of groups represented by Formula (2) to Formula (4),

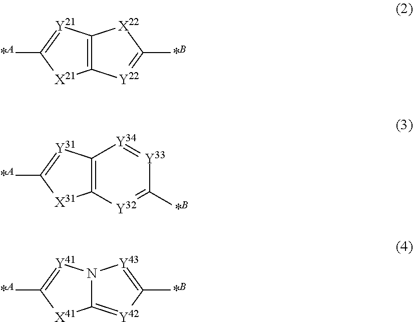

in Formula (2) to Formula (4), *^{A} and *^{B} each represent a bonding position,
in Formula (2), X²¹ and X²² each independently represent a sulfur atom, an oxygen atom, or a selenium atom, Y²¹ and Y²² each independently represent a nitrogen atom or —CR═, and R represents a hydrogen atom or a substituent, where, at least one of Y²¹ or Y²² represents a nitrogen atom,
in Formula (3), X³¹ represents a sulfur atom, an oxygen atom, or a selenium atom, Y³¹ to Y³⁴ each independently represent a nitrogen atom or —CR═, and R represents a hydrogen atom or a substituent,
in Formula (4), X⁴¹ represents a sulfur atom, an oxygen atom, or a selenium atom, Y⁴¹ to Y⁴³ each independently represent a nitrogen atom or —CR═, and R represents a hydrogen atom or a substituent.
   [15]

The compound according to [14], in which in Formula (1), a group represented by Ar¹¹ is any one of groups represented by Formula (A1) to Formula (A6),

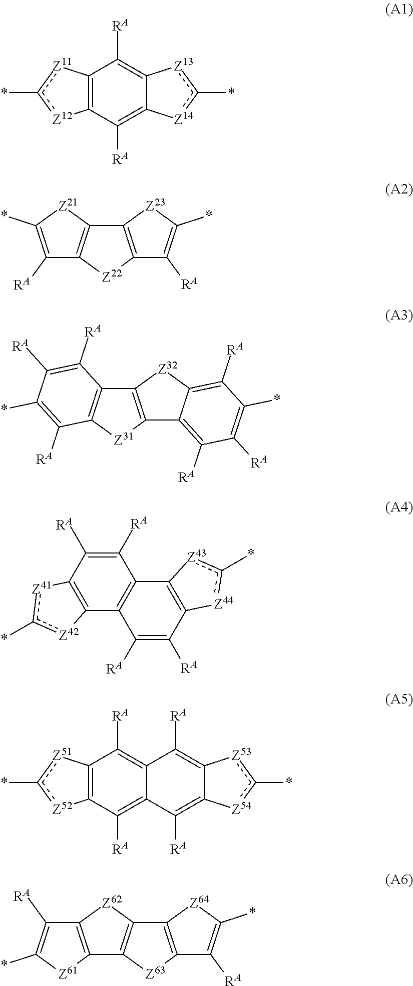

in Formulae (A1) to (A6), * represents a bonding position,
in Formula (A1), one of Z¹¹ or Z¹² represents a sulfur atom, an oxygen atom, or a selenium atom, and the other of Z¹¹ or Z¹² represents —CR═, one of Z¹³ or Z¹⁴ represents a sulfur atom, an oxygen atom, or a selenium atom, and the other of Z¹³ or Z¹⁴ represents —CR═, and R and R^{A} each independently represent a hydrogen atom or a substituent,
in Formula (A2), Z²¹ to Z²³ each independently represent a sulfur atom, an oxygen atom, or a selenium atom, and R^{A} represents a hydrogen atom or a substituent,
in Formula (A3), Z³¹ and Z³² each independently represent a sulfur atom, an oxygen atom, or a selenium atom, and R^{A} represents a hydrogen atom or a substituent,
in Formula (A4), one of Z⁴¹ or Z⁴² represents a sulfur atom, an oxygen atom, or a selenium atom, and the other of Z⁴¹ or Z⁴² represents —CR═, one of Z⁴³ or Z⁴⁴ represents a sulfur atom, an oxygen atom, or a selenium atom, and the other of Z⁴³ or Z⁴⁴ represents —CR═, and R and R^{A} each independently represent a hydrogen atom or a substituent,
in Formula (A5), one of Z⁵¹ or Z⁵² represents a sulfur atom, an oxygen atom, or a selenium atom, and the other of Z⁵¹ or Z⁵² represents —CR═, one of Z⁵³ or Z⁵⁴ represents a sulfur atom, an oxygen atom, or a selenium atom, and the other of Z⁵³ or Z⁵⁴ represents —CR═, and R and R^{A} each independently represent a hydrogen atom or a substituent,
in Formula (A6), Z⁶¹ to Z⁶⁴ each independently represent a sulfur atom, an oxygen atom, or a selenium atom, and R^{A} represents a hydrogen atom or a substituent.
   [16]

The compound according to [15], in which in Formula (A1), one of Z¹¹ or Z¹² represents a sulfur atom or an oxygen atom, and the other of Z¹¹ or Z¹² represents —CR═, and
one of Z¹³ or Z¹⁴ represents a sulfur atom or an oxygen atom, and the other of Z¹³ or Z¹⁴ represents —CR═,
in Formula (A2), Z²¹ to Z²³ each independently represent a sulfur atom or an oxygen atom,
in Formula (A3), Z³¹ and Z³² each independently represent a sulfur atom or an oxygen atom,
in Formula (A4), one of Z⁴¹ or Z⁴² represents a sulfur atom or an oxygen atom, and the other of Z⁴¹ or Z⁴² represents —CR═, and
one of Z⁴³ or Z⁴⁴ represents a sulfur atom or an oxygen atom, and the other of Z⁴³ or Z⁴ represents —CR═,
in Formula (A5), one of Z⁵¹ or Z⁵² represents a sulfur atom or an oxygen atom, and the other of Z⁵¹ or Z⁵² represents —CR═, and
one of Z⁵³ or Z⁵⁴ represents a sulfur atom or an oxygen atom, and the other of Z⁵³ or Z⁵⁴ represents —CR═, and
in Formula (A6), Z⁶¹ to Z⁶⁴ each independently represent a sulfur atom or an oxygen atom.
   [17]

The compound according to [15] or [16], in which in Formula (1), n11 is 1, and Ar¹¹ is a group represented by Formula (A5).

[18]

The compound according to any one of [14] to [17], in which in Formula (2), X²¹ and X²² each independently represent a sulfur atom or an oxygen atom,
in Formula (3), X³¹ represents a sulfur atom or an oxygen atom, and
in Formula (4), X⁴¹ represents a sulfur atom or an oxygen atom.
   [19]

The compound according to any one of [14] to [18], in which in Formula (1), Ar¹² and Ar¹³ each independently represent any one of groups represented by Formula (5) to Formula (13),

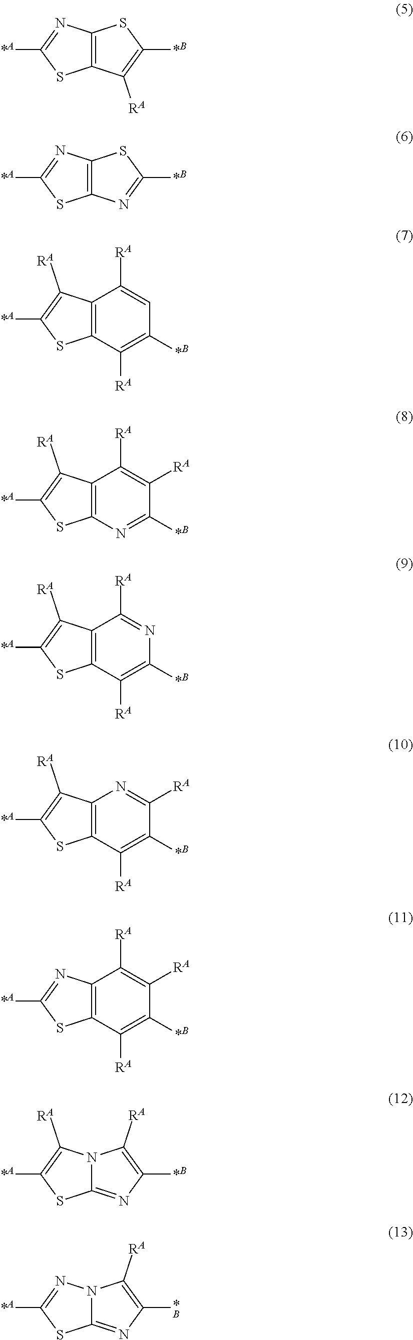

in Formula (5) to Formula (13), *A *B each represent a bonding position, and
R^{A} represents a hydrogen atom or a substituent.
   [20]

The compound according to any one of [14] to [19], in which the compound represented by Formula (1) has a molecular weight of 550 to 1200.

According to the present invention, it is possible to provide the photoelectric conversion element with an excellent photoelectric conversion efficiency.

In addition, according to the present invention, it is possible to provide the imaging element, the optical sensor, and the compound related to the photoelectric conversion element.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, suitable embodiments of a photoelectric conversion element of the present invention will be described.

In the present specification, examples of the halogen atom include a fluorine atom, a chlorine atom, a bromine atom, and an iodine atom.

In the present specification, in a case where a plurality of identical symbols indicating a kind or the number of groups are present in one formula (general formula), which indicates a chemical structure, contents of these plurality of identical symbols are independent of each other, and the contents of the identical symbols may be the same or different from each other unless otherwise specified.

In addition, in the present specification, the numerical range represented by “to” means a range including numerical values denoted before and after “to” as a lower limit value and an upper limit value.

In the present specification, a hydrogen atom may be a light hydrogen atom (an ordinary hydrogen atom) or a deuterium atom (a double hydrogen atom and the like).

### [Photoelectric Conversion Element]

The photoelectric conversion element according to an embodiment of the present invention includes a conductive film, a photoelectric conversion film, and a transparent conductive film in this order, in which the photoelectric conversion film contains a compound represented by Formula (1) (hereinafter, referred to as a “specific compound”).

The mechanism capable of solving the above problems by adopting such a configuration of the photoelectric conversion element according to the embodiment of the present invention is not always clear, but the present inventors have presumed as follows.

That is, the specific compound contains a predetermined linking group (Ar¹² and/or Ar¹³) between a mother nucleus, which is a fused polycyclic aromatic ring group (Ar¹¹) acting as a donor, and an aryl group or a heteroaryl group (Ar¹⁴ and/or Ar¹⁵) acting as an acceptor. The linking group is a fused polycyclic aromatic heterocyclic group having a structure in which predetermined 5-membered rings are fused, or a structure in which a 5-membered ring and a 6-membered ring are fused, and a bonding position with respect to a donor and/or an acceptor is also specified to be a predetermined position.

In this way, the donor is sandwiched between the acceptors, and the predetermined linking group as described above is further provided between the donor and the acceptor, thereby improving light absorption of the specific compound particularly at a wavelength of 400 to 550 nm. In addition, it is presumed that the above-described structure of the specific compound enables the improvement of charge transportability in the specific compound, between the specific compounds, or between the specific compound and another component, and an improvement to the photoelectric conversion efficiency of the photoelectric conversion element (in particular, the photoelectric conversion efficiency with respect to light having a wavelength of 400 to 550 nm).

The electric field strength dependence of the photoelectric conversion efficiency in the photoelectric conversion element according to the embodiment of the present invention is further suppressed. It is presumed to be based on the fact that the linking group included in the specific compound enables the specific compound to have a packing structure preferable for charge transport in the photoelectric conversion film and to maintain favorable charge transportability even under a low voltage.

Hereinafter, the fact that the photoelectric conversion efficiency of the photoelectric conversion element is more excellent and/or that the electric field strength dependence of the photoelectric conversion efficiency is further suppressed is also referred to as “the effect of the present invention is more excellent”.

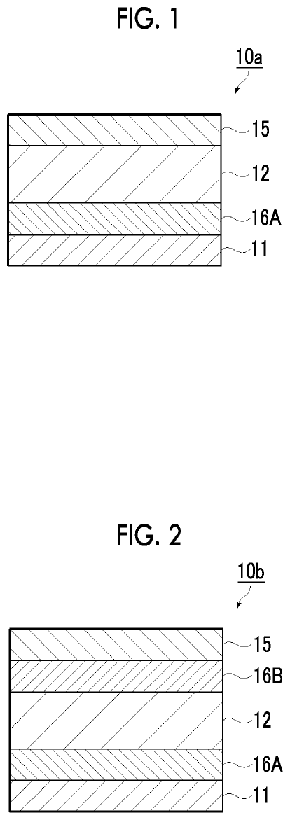

FIG. 1 is a schematic cross-sectional view of one embodiment of a photoelectric conversion element of the present invention.

A photoelectric conversion element 10a illustrated in FIG. 1 has a configuration in which a conductive film (hereinafter, also referred to as a lower electrode) 11 functioning as a lower electrode, an electron blocking film 16A, a photoelectric conversion film 12 containing the specific compound described later, and a transparent conductive film (hereinafter, also referred to as an upper electrode) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a hole blocking film 16B, and an upper electrode 15 are laminated on a lower electrode 11 in this order. The lamination order of the electron blocking film 16A, the photoelectric conversion film 12, and the hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of the performance and power consumption, the applied voltage is more preferably 1×10⁻⁴ to 1×10⁷ V/cm, and still more preferably 1×10⁻³ to 5×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

### [Photoelectric Conversion Film]

The photoelectric conversion film is a film containing a specific compound. Hereinafter, the specific compound will be described in detail.

### <Compound (Specific Compound) Represented by Formula (1)>

The specific compound is a compound represented by Formula (1).

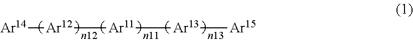

In Formula (1), n11 represents 1 or 2.

In particular, from the viewpoint that the effect of the present invention is more excellent, in a case where Ar¹¹ is a fused polycyclic aromatic ring group composed of four rings (for example, Ar¹¹ is a group represented by Formula (A3) to Formula (A6) described later), n11 is preferably 1.

In Formula (1), n12 and n13 each independently represent 0 or 1. Here, at least one of n12 or n13 represents 1.

It is preferable that both n12 and n13 are 1.

In Formula (1), Ar¹¹ represents a fused polycyclic aromatic ring group consisting of a combination of one or more (for example, one to four) aromatic rings selected from the group consisting of a thiophene ring, a benzene ring, a furan ring, and a selenophene ring.

In particular, Ar¹¹ preferably represents a fused polycyclic aromatic ring group consisting of a combination of one or more (for example, one to three) aromatic rings selected from the group consisting of a thiophene ring, a benzene ring, and a furan ring.

Here, the fused polycyclic aromatic ring group has three or four rings.

At least one ring of three or four rings constituting the fused polycyclic aromatic ring group is preferably a ring other than a benzene ring, and at least two rings are more preferably rings other than a benzene ring.

The fused polycyclic aromatic ring group preferably contains at least one (for example, one to four, and preferably two) thiophene ring or furan ring, and more preferably contains a thiophene ring.

The fused polycyclic aromatic ring group may or may not have a substituent. As the substituent, a halogen atom (a fluorine atom or other atoms) is preferable.

For example, one to six substituents are contained in the fused polycyclic aromatic ring group.

In a case where n 11 represents 2, two Ar¹¹ 's are preferably identical.

In particular, Ar¹¹ in Formula (1) is preferably any one of groups represented by Formula (A1) to Formula (A6), more preferably any one of groups represented by any one of Formula (A3) to Formula (A6), and still more preferably a group represented by Formula (A5).

In particular, in Formula (1), it is particularly preferable that n11 is 1, and Ar¹¹ is a group represented by Formula (A5).

In addition, it is also preferable that (Ar¹¹)ₙ₁₁ in Formula (1) is a group composed of the same groups that are represented by the same Formulae among Formula (A1) to Formula (A6) and that are linked to each other. For example, a group composed of two groups that are represented by Formula (A1) and that are linked to each other is also preferable.

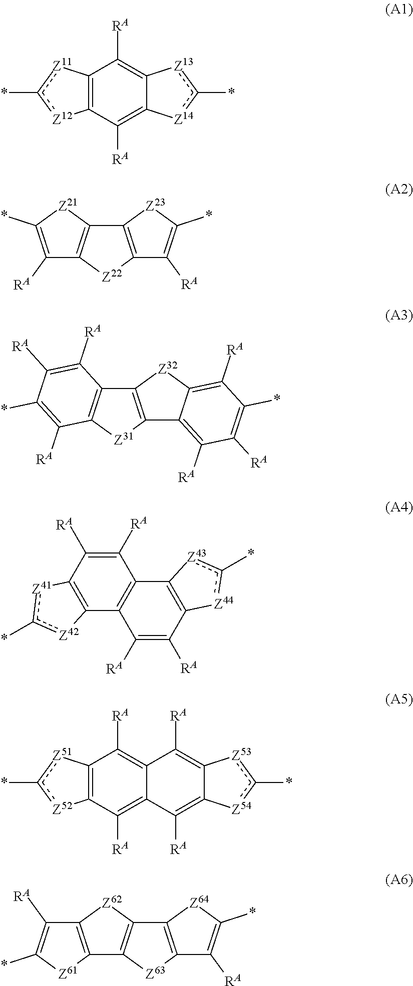

In Formulae (A1) to (A6), * represents a bonding position.

In Formula (A1), one of Z¹¹ or Z¹² represents a sulfur atom (—S—), an oxygen atom (—O—), or a selenium atom (—Se—), and the other of Z¹¹ or Z¹² represents —CR═. One of Z¹¹ or Z¹² preferably represents a sulfur atom or an oxygen atom, and the other of Z¹¹ or Z¹² preferably represents —CR═.

One of Z¹³ or Z¹⁴ represents a sulfur atom, an oxygen atom, or a selenium atom, and the other of Z¹³ or Z¹⁴ represents —CR═. One of Z¹³ or Z¹⁴ preferably represents a sulfur atom or an oxygen atom, and the other of Z¹³ or Z¹⁴ preferably represents —CR═.

R and R^{A} each independently represent a hydrogen atom or a substituent, and are each preferably a hydrogen atom.

The substituent, which may be represented by R and R^{A}, is preferably a halogen atom (a fluorine atom or other atoms) or an alkyl group, which may further have a halogen atom (for example, one or two carbon atoms), and a halogen atom (a fluorine atom or other atoms) is more preferable.

In Formula (A2), Z²¹ to Z²³ each independently represent a sulfur atom, an oxygen atom, or a selenium atom, and preferably represent a sulfur atom or an oxygen atom.

R^{A} represents a hydrogen atom or a substituent, and is preferably a hydrogen atom.

As a substituent which may be represented by R^{A}, a halogen atom (a fluorine atom or other atoms) is preferable.

In Formula (A3), Z³¹ and Z³² each independently represent a sulfur atom, an oxygen atom, or a selenium atom, and preferably represent a sulfur atom or an oxygen atom.

R^{A} represents a hydrogen atom or a substituent, and is preferably a hydrogen atom.

As a substituent which may be represented by R^{A}, a halogen atom (a fluorine atom or other atoms) is preferable.

In Formula (A4), one of Z⁴¹ or Z⁴² represents a sulfur atom, an oxygen atom, or a selenium atom, and the other of Z⁴¹ or Z⁴² represents —CR═. One of Z⁴¹ or Z⁴² preferably represents a sulfur atom or an oxygen atom, and the other of Z⁴¹ or Z⁴² preferably represents —CR═.

One of Z⁴³ or Z⁴⁴ represents a sulfur atom, an oxygen atom, or a selenium atom, and the other of Z⁴³ or Z⁴⁴ represents —CR═. One of Z⁴³ or Z⁴⁴ preferably represents a sulfur atom or an oxygen atom, and the other of Z⁴³ or Z⁴⁴ preferably represents —CR═.

R and R^{A} each independently represent a hydrogen atom or a substituent, and are each preferably a hydrogen atom.

As a substituent which may be represented by R or R^{A}, a halogen atom (a fluorine atom or other atoms) is preferable.

In Formula (A5), one of Z⁵¹ or Z⁵² represents a sulfur atom, an oxygen atom, or a selenium atom, and the other of Z⁵¹ or Z⁵² represents —CR═. One of Z⁵¹ or Z⁵² preferably represents a sulfur atom or an oxygen atom, and the other of Z⁵¹ or Z⁵² preferably represents —CR═.

One of Z⁵³ or Z⁵⁴ represents a sulfur atom, an oxygen atom, or a selenium atom, and the other of Z⁵³ or Z⁵⁴ represents —CR═. It is preferable that one of Z⁵³ and Z⁵⁴ represents a sulfur atom or an oxygen atom, and the other of Z⁵³ and Z⁵⁴ represents —CR═.

R and R^{A} each independently represent a hydrogen atom or a substituent, and are each preferably a hydrogen atom.

As a substituent which may be represented by R or R^{A}, a halogen atom (a fluorine atom or other atoms) is preferable.

In Formula (A6), Z⁶¹ to Z⁶⁴ each independently represent a sulfur atom, an oxygen atom, or a selenium atom, and preferably represent a sulfur atom or an oxygen atom.

R^{A} represents a hydrogen atom or a substituent.

As a substituent which may be represented by R^{A}, a halogen atom (a fluorine atom or other atoms) is preferable.

In Formula (1), Ar¹⁴ and Ar¹⁵ each independently represent an aryl group which may have a substituent or a heteroaryl group which may have a substituent.

The aryl group may be monocyclic or polycyclic, and the number of ring member atoms is preferably 6 to 15. The aryl group is preferably a phenyl group, a naphthyl group, or an anthracenyl group, and more preferably a phenyl group.

The heteroaryl group may be monocyclic or polycyclic, and the number of ring member atoms is preferably 5 to 15. The number of heteroatoms contained in the aryl group is preferably 1 to 5, and more preferably 1. Examples of the heteroatom include a nitrogen atom, an oxygen atom, a sulfur atom, and a selenium atom. As the heteroaryl group, a pyridinyl group, a pyrazyl group, a pyrimidyl group, a pyridazyl group, a triazyl group, a quinolyl group, a quinoxalyl group, a quinazolyl group, a phthalazyl group, a cinnolyl group, an isoquinolyl group, a pteridyl group, an acridyl group, a phenadyl group, a phenanthrolyl group, a tetrazolyl group, a pyrazolyl group, an imidazolyl group, or a thiazolyl group is preferable.

As a substituent which the aryl group or heteroaryl group may have, an alkyl group which may further have a substituent or a halogen atom (a fluorine atom or other atoms) is preferable.

The alkyl group may be linear or branched, and preferably has one to three carbon atoms. Examples of the substituent which the alkyl group may further have include a halogen atom (a fluorine atom or other atoms). For example, the alkyl group is preferably a halogenated alkyl group (a fluoroalkyl group or other groups), and more preferably a perhalogenated alkyl group (a perfluoroalkyl group or other groups).

In particular, Ar¹⁴ and Ar¹⁵ are each independently preferably an aryl group having a halogenated alkyl group or a halogen atom as a substituent, or a heteroaryl group which may have a substituent.

Ar¹⁴ and Ar¹⁵ may be the same as or different from each other, and are preferably the same as each other.

In Formula (1), Ar¹² and Ar¹³ each independently represent any one of groups represented by Formula (2) to Formula (4).

Ar¹² and Ar¹³ may be the same as or different from each other, and are preferably the same as each other.

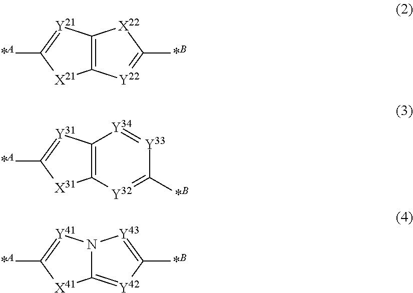

In Formula (2) to Formula (4), *^{A} and *^{B} each represent a bonding position.

In Formula (2) to Formula (4), in *^{A} and *^{B}, *^{A} may be a bonding position at the (Ar¹¹)ₙ₁₁ side, or *^{B} may be the bonding position at the (Ar¹¹)₁₁ side.

In particular, in Formula (3), *^{A} is preferably the bonding position at the (Ar¹¹)₁₁ side.

In Formula (2), X²¹ and X²² each independently represent a sulfur atom, an oxygen atom, or a selenium atom, and are each preferably a sulfur atom or an oxygen atom.

Y²¹ and Y²² each independently represent a nitrogen atom (—N═) or —CR═. R represents a hydrogen atom or a substituent.

Y²¹ and Y²² are each independently preferably a nitrogen atom or —CH═.

As a substituent which may be represented by R, a halogen atom (a fluorine atom or other atoms) is preferable.

Here, at least one of Y²¹ or Y²² represents a nitrogen atom.

Only Y²¹ of Y²¹ and Y²² may be a nitrogen atom, only Y²² may be a nitrogen atom, or both may be nitrogen atoms.

In Formula (3), X³¹ represents an oxygen atom, a sulfur atom, or a selenium atom, and a sulfur atom or an oxygen atom is preferable.

Y³¹ to Y³⁴ each independently represent a nitrogen atom or —CR═. R represents a hydrogen atom or a substituent.

Y³¹ to Y³⁴ are each independently preferably a nitrogen atom or —CH═.

As a substituent which may be represented by R, a halogen atom (a fluorine atom or other atoms) is preferable.

None of Y³¹ to Y³⁴ or one to four thereof may be a nitrogen atom, none of Y³¹ to Y³⁴ or one of Y³¹, Y³², Y³³, or Y³⁴ is preferably a nitrogen atom, and one of Y³¹, Y³², Y³³, or Y³⁴ is more preferably a nitrogen atom.

In Formula (4), X⁴¹ represents an oxygen atom, a sulfur atom, or a selenium atom, and a sulfur atom or an oxygen atom is preferable.

Y⁴¹ to Y⁴³ each independently represent a nitrogen atom or —CR═. R represents a hydrogen atom or a substituent.

Y⁴¹ to Y⁴³ are each independently preferably a nitrogen atom or —CH═.

As a substituent which may be represented by R, a halogen atom (a fluorine atom or other atoms) is preferable.

None of Y⁴¹ to Y⁴³ or one to three thereof may be a nitrogen atom, and one or two of Y⁴¹ to Y⁴³ are preferably a nitrogen atom.

Ar¹² and Ar¹³ each independently preferably represent any one of groups represented by Formula (5) to Formula (13), and more preferably represent any one of the groups represented by Formula (5), Formula (6), and Formula (8) to Formula (13).

In particular, Ar¹¹ is a group represented by Formula (A5), and Ar¹² and Ar¹³ are each independently preferably any one of groups represented by Formula (5), Formula (6), and Formula (8) to Formula (13).

The groups represented by Formula (5) and Formula (6) are suitable forms of the group represented by Formula (2), and the groups represented by Formula (7) to Formula (11) are suitable forms of the group represented by Formula (3), and the groups represented by Formula (12) and Formula (13) are suitable forms of the group represented by Formula (4).

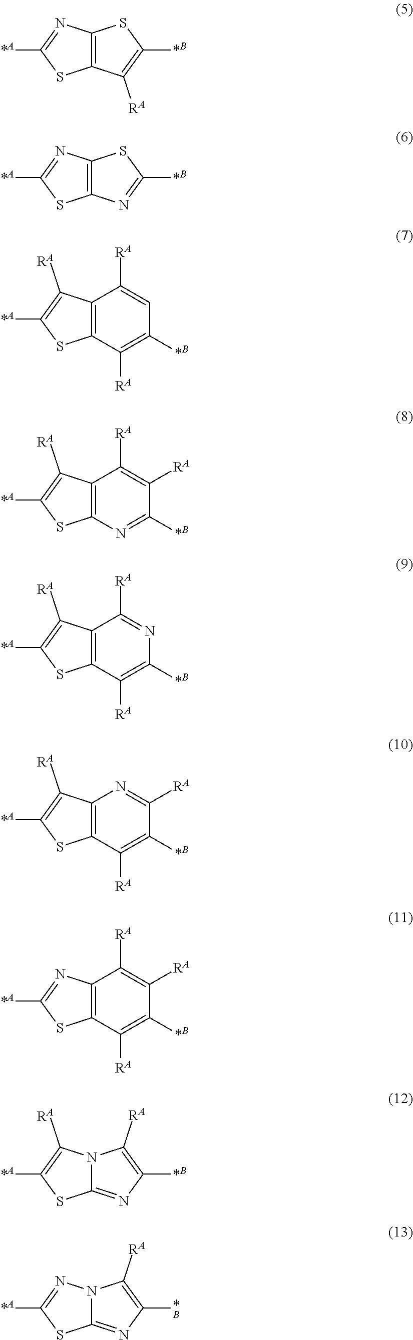

In Formula (5) to Formula (13), *^{A} and *^{B} each represent a bonding position.

In Formula (5) to Formula (13), in *^{A} and *^{B}, *^{A} may be a bonding position at the (Ar¹¹)ₙ₁₁ side, or *B may be the bonding position at the (Ar¹¹)ₙ₁₁ side. In particular, in Formula (7) to Formula (11), *^{A} is preferably the bonding position at the (Ar¹¹)ₙ₁₁ side.

R^{A} represents a hydrogen atom or a substituent, and is preferably a hydrogen atom.

As a substituent which may be represented by R^{A}, a halogen atom (a fluorine atom or other atoms) is preferable.

Specific examples of the specific compound will be described below.

In each of the following structural formulae illustrating specific examples of the specific compounds, examples of possible combination forms of individual groups of Ar¹⁴ to Ar¹⁵, n12, and n13 are illustrated in the following Tables.

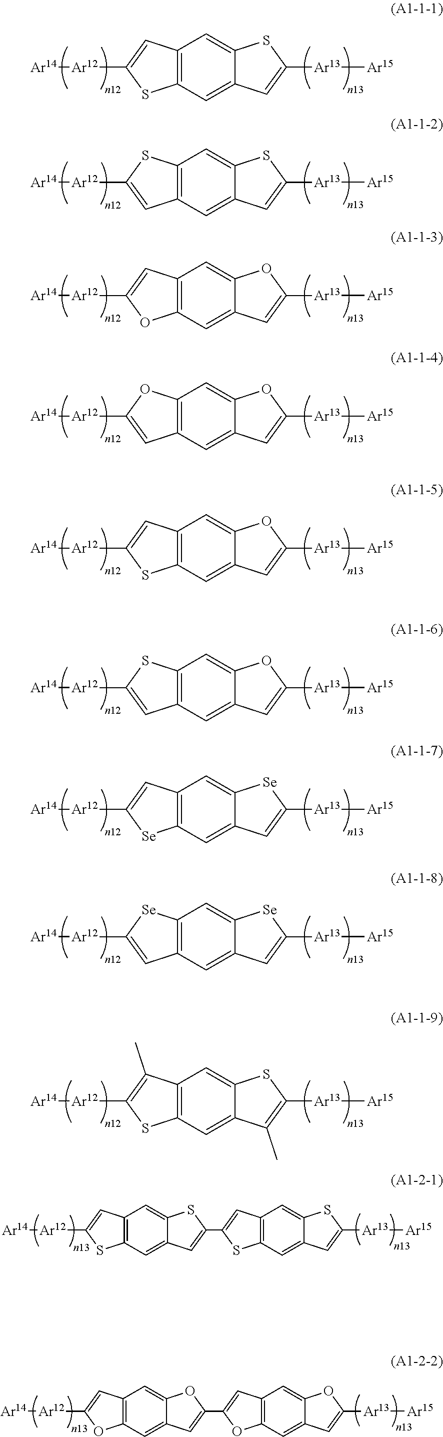

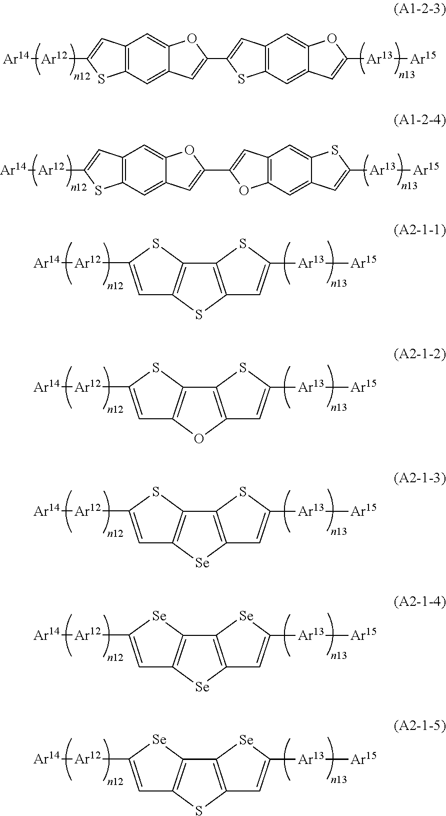

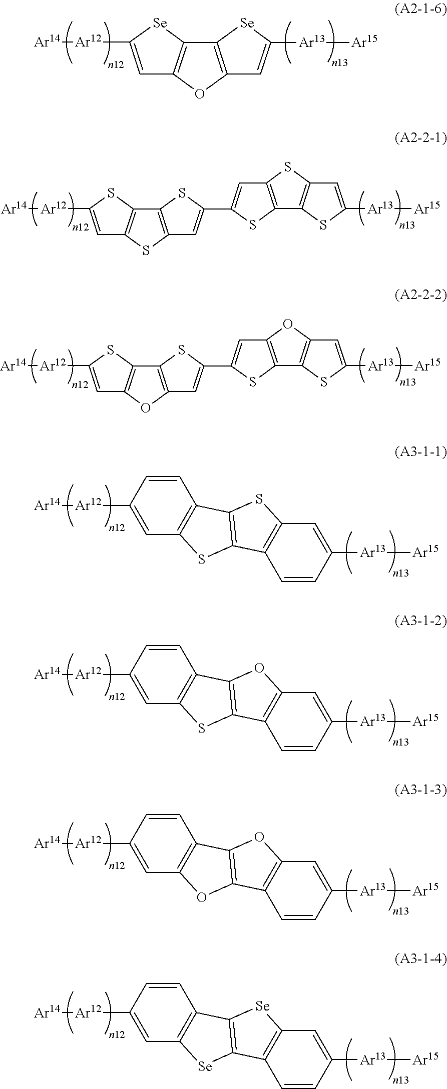

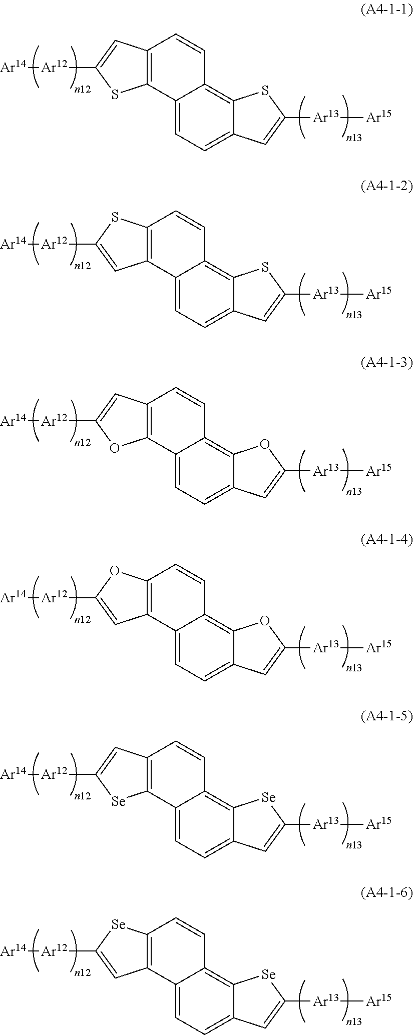

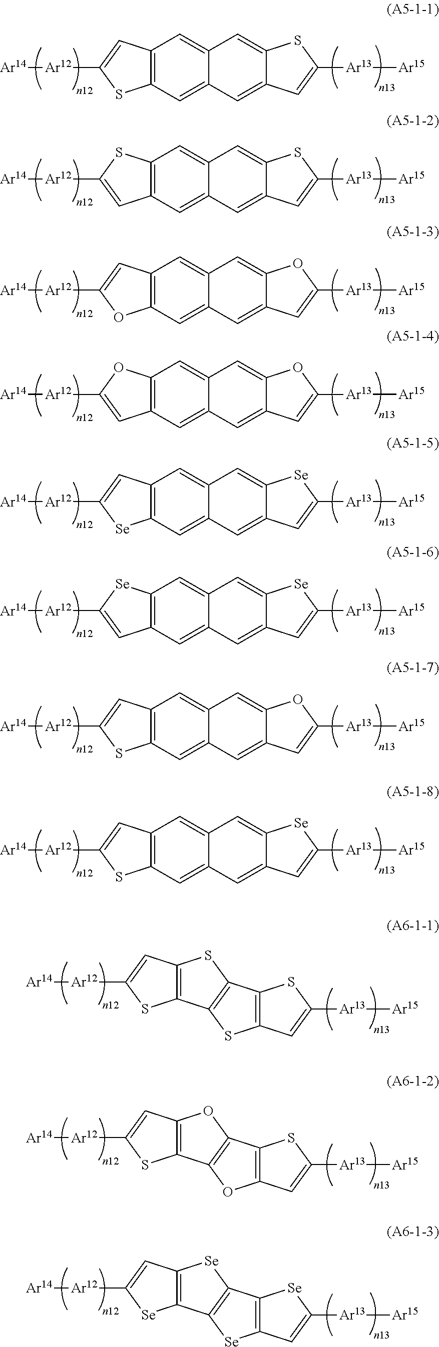

Examples of possible combination forms of individual groups or individual numerical values of Ar¹⁴ to Ar¹⁵, n12, and n13 in each of the above structural formulae are illustrated in Tables below.

In Tables below, * indicates a bonding position, and Ph indicates a phenyl group.

In Tables below, a bonding position (*) at the left side of each group illustrated in the “Ar¹²” column is a bonding position at the Ar¹⁴ side, and a bonding position (*) at the right side is a bonding position at the center side (which is a side where a group corresponding to (Ar¹¹)ₙ₁₁ in Formula (1) is present). A bonding position (*) at the right side of each group illustrated in the “Ar¹³” column is a bonding position at the Ar¹⁵ side, and a bonding position (*) at the left side is a bonding position at the center side (which is a side where a group corresponding to (Ar¹¹)ₙ₁₁ in Formula (1) is present).

TABLE 1

| No | n12 | Ar¹² | n13 | Ar¹³ | Ar¹⁴ | Ar¹⁵ |
|---|---|---|---|---|---|---|
| 1 | 1 | 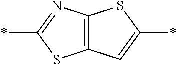 [SMILES: *c1cc2sc(*)nc2s1] | 1 | 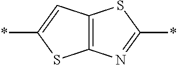 [SMILES: *c1cc2sc(*)nc2s1] | Ph | Ph |
| 2 | 2 | 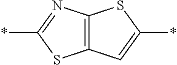 [SMILES: *c1cc2sc(*)nc2s1] | 1 | 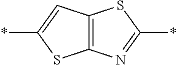 [SMILES: *c1cc2sc(*)nc2s1] | Ph | Ph |
| 3 | 1 | 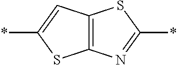 [SMILES: *c1cc2sc(*)nc2s1] | 1 | 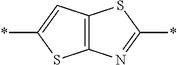 [SMILES: *c1cc2sc(*)nc2s1] | Ph | Ph |
| 4 | 1 | 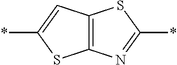 [SMILES: *c1cc2sc(*)nc2s1] | 1 | 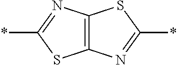 [SMILES: *c1nc2sc(*)nc2s1] | Ph | Ph |
| 5 | 1 | 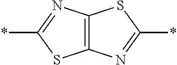 [SMILES: *c1nc2sc(*)nc2s1] | 1 | 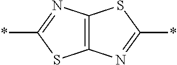 [SMILES: *c1nc2sc(*)nc2s1] | Ph | Ph |
| 6 | 1 | 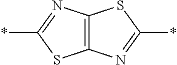 [SMILES: *c1nc2sc(*)nc2s1] | 1 | 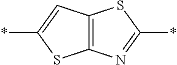 [SMILES: *c1cc2sc(*)nc2s1] | Ph | Ph |
| 7 | 1 | 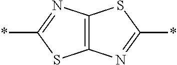 [SMILES: *c1nc2sc(*)nc2s1] | 1 | 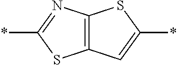 [SMILES: *c1cc2sc(*)nc2s1] | Ph | Ph |
| 8 | 1 | 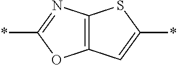 [SMILES: *c1nc2sc(*)cc2o1] | 1 | 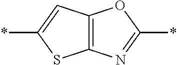 [SMILES: *c1nc2sc(*)cc2o1] | Ph | Ph |
| 9 | 1 | 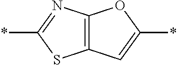 [SMILES: *c1cc2sc(*)nc2o1] | 1 | 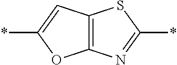 [SMILES: *c1cc2sc(*)nc2o1] | Ph | Ph |
| 10 | 1 | 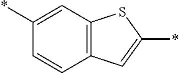 [SMILES: *c1ccc2cc(*)sc2c1] | 1 | 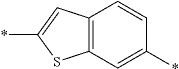 [SMILES: *c1ccc2cc(*)sc2c1] | Ph | Ph |
| 11 | 1 | 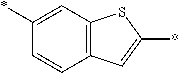 [SMILES: *c1ccc2cc(*)sc2c1] | 1 | 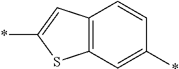 [SMILES: *c1ccc2cc(*)sc2c1] | 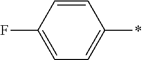 [SMILES: *c1ccc(F)cc1] | 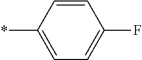 [SMILES: *c1ccc(F)cc1] |
| 12 | 1 | 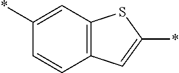 [SMILES: *c1ccc2cc(*)sc2c1] | 1 | 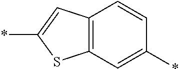 [SMILES: *c1ccc2cc(*)sc2c1] | 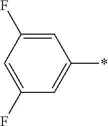 [SMILES: *c1cc(F)cc(F)c1] | 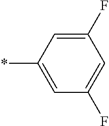 [SMILES: *c1cc(F)cc(F)c1] |
| 13 | 1 | 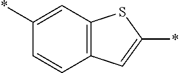 [SMILES: *c1ccc2cc(*)sc2c1] | 1 | 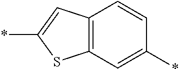 [SMILES: *c1ccc2cc(*)sc2c1] | 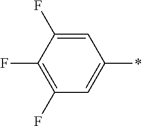 [SMILES: *c1cc(F)c(F)c(F)c1] | 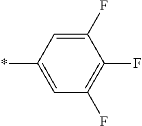 [SMILES: *c1cc(F)c(F)c(F)c1] |
| 14 | 1 | 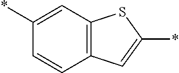 [SMILES: *c1ccc2cc(*)sc2c1] | 1 | 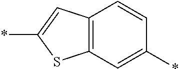 [SMILES: *c1ccc2cc(*)sc2c1] | 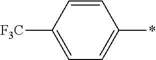 [SMILES: *c1ccc(C(F)(F)F)cc1] | 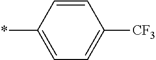 [SMILES: *c1ccc(C(F)(F)F)cc1] |
| 15 | 1 | 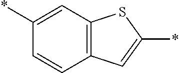 [SMILES: *c1ccc2cc(*)sc2c1] | 1 | 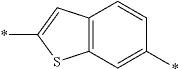 [SMILES: *c1ccc2cc(*)sc2c1] | 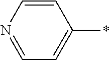 [SMILES: *c1ccncc1] | 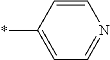 [SMILES: *c1ccncc1] |
| 16 | 1 | 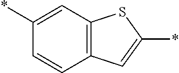 [SMILES: *c1ccc2cc(*)sc2c1] | 1 | 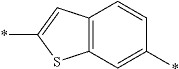 [SMILES: *c1ccc2cc(*)sc2c1] | 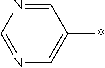 [SMILES: *c1cncnc1] | 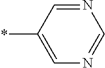 [SMILES: *c1cncnc1] |

TABLE 2

| No. | n12 | Ar¹² | n13 | Ar¹³ | Ar¹⁴ | Ar¹⁵ |
|---|---|---|---|---|---|---|
| 17 | 1 | 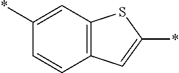 [SMILES: *c1ccc2cc(*)sc2c1] | 1 | 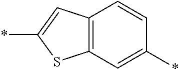 [SMILES: *c1ccc2cc(*)sc2c1] | 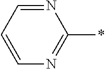 [SMILES: *c1ncccn1] | 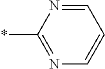 [SMILES: *c1ncccn1] |
| 18 | 1 | 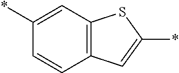 [SMILES: *c1ccc2cc(*)sc2c1] | 1 | 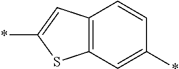 [SMILES: *c1ccc2cc(*)sc2c1] | 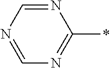 [SMILES: *c1ncncn1] | 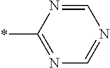 [SMILES: *c1ncncn1] |
| 19 | 1 | 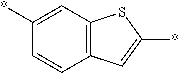 [SMILES: *c1ccc2cc(*)sc2c1] | 1 | 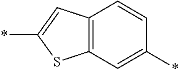 [SMILES: *c1ccc2cc(*)sc2c1] | 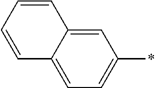 [SMILES: *c1ccc2ccccc2c1] | 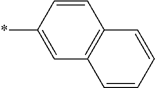 [SMILES: *c1ccc2ccccc2c1] |
| 20 | 1 | 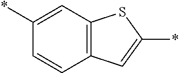 [SMILES: *c1ccc2cc(*)sc2c1] | 1 | 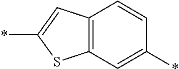 [SMILES: *c1ccc2cc(*)sc2c1] | 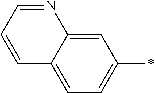 [SMILES: *c1ccc2cccnc2c1] | 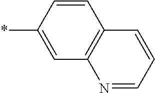 [SMILES: *c1ccc2cccnc2c1] |
| 21 | 1 | 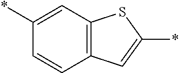 [SMILES: *c1ccc2cc(*)sc2c1] | 1 | 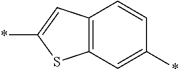 [SMILES: *c1ccc2cc(*)sc2c1] | 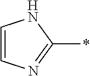 [SMILES: *c1ncc[nH]1] | 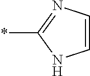 [SMILES: *c1ncc[nH]1] |
| 22 | 1 | 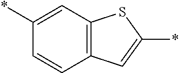 [SMILES: *c1ccc2cc(*)sc2c1] | 1 | 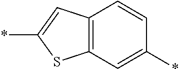 [SMILES: *c1ccc2cc(*)sc2c1] | 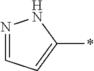 [SMILES: *c1ccn[nH]1] | 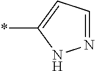 [SMILES: *c1ccn[nH]1] |
| 23 | 1 | 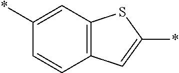 [SMILES: *c1ccc2cc(*)sc2c1] | 1 | 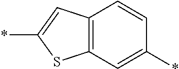 [SMILES: *c1ccc2cc(*)sc2c1] | 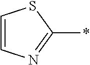 [SMILES: *c1nccs1] | 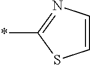 [SMILES: *c1nccs1] |
| 24 | 1 | 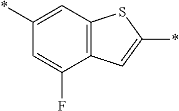 [SMILES: *c1cc(F)c2cc(*)sc2c1] | 1 | 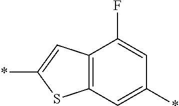 [SMILES: *c1cc(F)c2cc(*)sc2c1] | Ph | Ph |
| 25. | 1 | 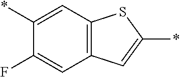 [SMILES: *c1cc2cc(F)c(*)cc2s1] | 1 | 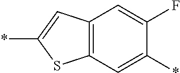 [SMILES: *c1cc2cc(F)c(*)cc2s1] | Ph | Ph |
| 26 | 1 | 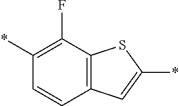 [SMILES: *c1cc2ccc(*)c(F)c2s1] | 1 | 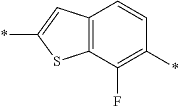 [SMILES: *c1cc2ccc(*)c(F)c2s1] | Ph | Ph |
| 27 | 1 | 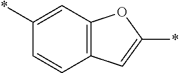 [SMILES: *c1ccc2cc(*)oc2c1] | 1 | 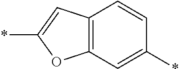 [SMILES: *c1ccc2cc(*)oc2c1] | Ph | Ph |
| 28 | 1 | 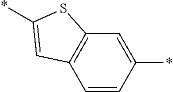 [SMILES: *c1ccc2cc(*)sc2c1] | 1 | 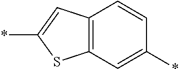 [SMILES: *c1ccc2cc(*)sc2c1] | Ph | Ph |
| 29 | 1 | 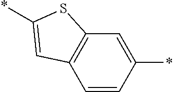 [SMILES: *c1ccc2cc(*)sc2c1] | 1 | 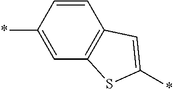 [SMILES: *c1ccc2cc(*)sc2c1] | Ph | Ph |
| 30 | 1 | 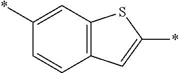 [SMILES: *c1ccc2cc(*)sc2c1] | 1 | 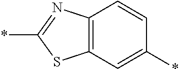 [SMILES: *c1ccc2nc(*)sc2c1] | Ph | Ph |
| 31 | 1 | 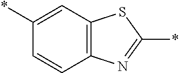 [SMILES: *c1ccc2nc(*)sc2c1] | 1 | 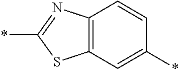 [SMILES: *c1ccc2nc(*)sc2c1] | Ph | Ph |

TABLE 3

| No | n12 | Ar¹² | n13 | Ar¹³ | Ar¹⁴ | Ar¹⁵ |
|---|---|---|---|---|---|---|
| 32 | 1 | 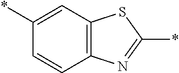 [SMILES: *c1ccc2nc(*)sc2c1] | 1 | 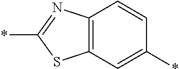 [SMILES: *c1ccc2nc(*)sc2c1] | 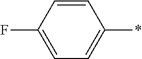 [SMILES: *c1ccc(F)cc1] | 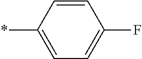 [SMILES: *c1ccc(F)cc1] |
| 33 | 1 | 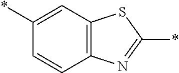 [SMILES: *c1ccc2nc(*)sc2c1] | 1 | 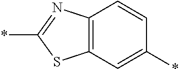 [SMILES: *c1ccc2nc(*)sc2c1] | 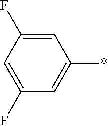 [SMILES: *c1cc(F)cc(F)c1] | 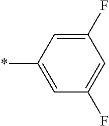 [SMILES: *c1cc(F)cc(F)c1] |
| 34 | 1 | 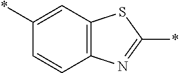 [SMILES: *c1ccc2nc(*)sc2c1] | 1 | 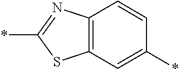 [SMILES: *c1ccc2nc(*)sc2c1] | 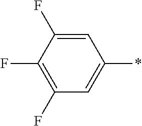 [SMILES: *c1cc(F)c(F)c(F)c1] | 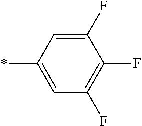 [SMILES: *c1cc(F)c(F)c(F)c1] |
| 35 | 1 | 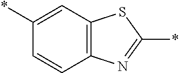 [SMILES: *c1ccc2nc(*)sc2c1] | 1 | 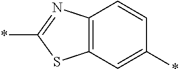 [SMILES: *c1ccc2nc(*)sc2c1] | 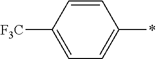 [SMILES: *c1ccc(C(F)(F)F)cc1] | 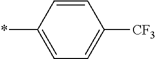 [SMILES: *c1ccc(C(F)(F)F)cc1] |
| 36 | 1 | 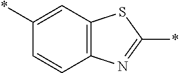 [SMILES: *c1ccc2nc(*)sc2c1] | 1 | 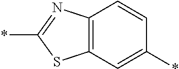 [SMILES: *c1ccc2nc(*)sc2c1] | 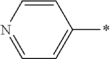 [SMILES: *c1ccncc1] | 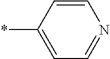 [SMILES: *c1ccncc1] |
| 37 | 1 | 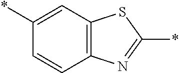 [SMILES: *c1ccc2nc(*)sc2c1] | 1 | 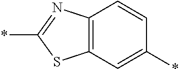 [SMILES: *c1ccc2nc(*)sc2c1] | 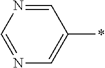 [SMILES: *c1cncnc1] | 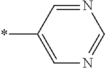 [SMILES: *c1cncnc1] |
| 38 | 1 | 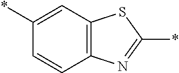 [SMILES: *c1ccc2nc(*)sc2c1] | 1 | 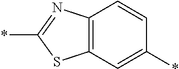 [SMILES: *c1ccc2nc(*)sc2c1] | 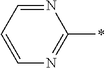 [SMILES: *c1ncccn1] | 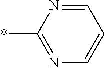 [SMILES: *c1ncccn1] |
| 39 | 1 | 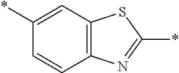 [SMILES: *c1ccc2nc(*)sc2c1] | 1 | 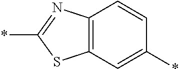 [SMILES: *c1ccc2nc(*)sc2c1] | 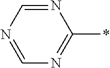 [SMILES: *c1ncncn1] | 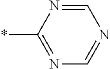 [SMILES: *c1ncncn1] |
| 40 | 1 | 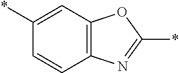 [SMILES: *c1ccc2nc(*)oc2c1] | 1 | 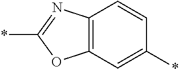 [SMILES: *c1ccc2nc(*)oc2c1] | Ph | Ph |
| 41 | 1 | 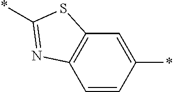 [SMILES: *c1ccc2nc(*)sc2c1] | 1 | 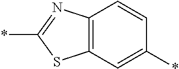 [SMILES: *c1ccc2nc(*)sc2c1] | Ph | Ph |
| 42 | 1 | 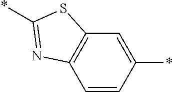 [SMILES: *c1ccc2nc(*)sc2c1] | 1 | 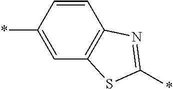 [SMILES: *c1ccc2nc(*)sc2c1] | Ph | Ph |
| 43 | 1 | 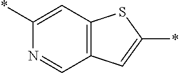 [SMILES: *c1cc2sc(*)cc2cn1] | 1 | 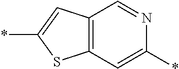 [SMILES: *c1cc2sc(*)cc2cn1] | Ph | Ph |
| 44 | 1 | 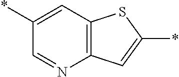 [SMILES: *c1cnc2cc(*)sc2c1] | 1 | 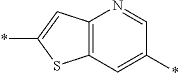 [SMILES: *c1cnc2cc(*)sc2c1] | Ph | Ph |
| 45 | 1 | 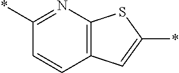 [SMILES: *c1ccc2cc(*)sc2n1] | 1 | 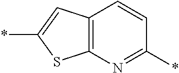 [SMILES: *c1ccc2cc(*)sc2n1] | Ph | Ph |
| 46 | 1 | 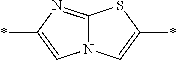 [SMILES: *c1cn2cc(*)sc2n1] | 1 | 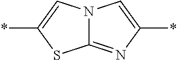 [SMILES: *c1cn2cc(*)sc2n1] | Ph | Ph |
| 47 | 1 | 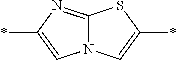 [SMILES: *c1cn2cc(*)sc2n1] | 1 | 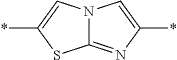 [SMILES: *c1cn2cc(*)sc2n1] | 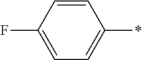 [SMILES: *c1ccc(F)cc1] | 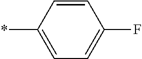 [SMILES: *c1ccc(F)cc1] |

TABLE 4

| No | n12 | Ar¹² | n13 | Ar¹³ | Ar¹⁴ | Ar¹⁵ |
|---|---|---|---|---|---|---|
| 48 | 1 | 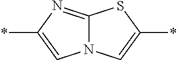 [SMILES: *c1cn2cc(*)sc2n1] | 1 | 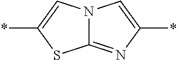 [SMILES: *c1cn2cc(*)sc2n1] | 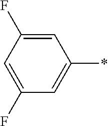 [SMILES: *c1cc(F)cc(F)c1] | 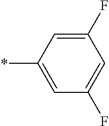 [SMILES: *c1cc(F)cc(F)c1] |
| 49 | 1 | 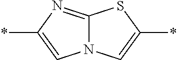 [SMILES: *c1cn2cc(*)sc2n1] | 1 | 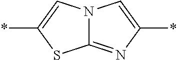 [SMILES: *c1cn2cc(*)sc2n1] | 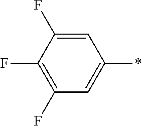 [SMILES: *c1cc(F)c(F)c(F)c1] | 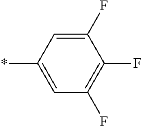 [SMILES: *c1cc(F)c(F)c(F)c1] |
| 50 | 1 | 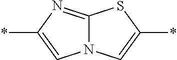 [SMILES: *c1cn2cc(*)sc2n1] | 1 | 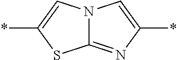 [SMILES: *c1cn2cc(*)sc2n1] | 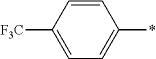 [SMILES: *c1ccc(C(F)(F)F)cc1] | 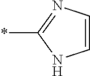 [SMILES: *c1ncc[nH]1] |
| 51 | 1 | 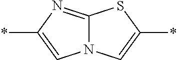 [SMILES: *c1cn2cc(*)sc2n1] | 1 | 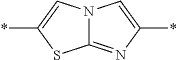 [SMILES: *c1cn2cc(*)sc2n1] | 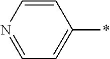 [SMILES: *c1ccncc1] | 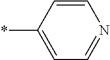 [SMILES: *c1ccncc1] |
| 52 | 1 | 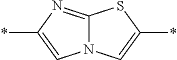 [SMILES: *c1cn2cc(*)sc2n1] | 1 | 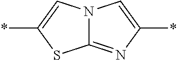 [SMILES: *c1cn2cc(*)sc2n1] | 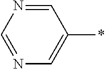 [SMILES: *c1cncnc1] | 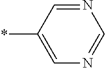 [SMILES: *c1cncnc1] |
| 53 | 1 | 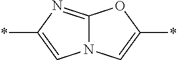 [SMILES: *c1cn2cc(*)oc2n1] | 1 | 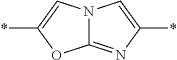 [SMILES: *c1cn2cc(*)oc2n1] | Ph | Ph |
| 54 | 1 | 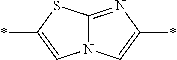 [SMILES: *c1cn2cc(*)sc2n1] | 1 | 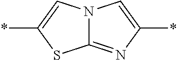 [SMILES: *c1cn2cc(*)sc2n1] | Ph | Ph |
| 55 | 1 | 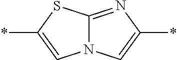 [SMILES: *c1cn2cc(*)sc2n1] | 1 | 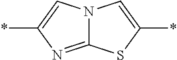 [SMILES: *c1cn2cc(*)sc2n1] | Ph | Ph |
| 56 | 1 | 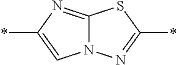 [SMILES: *c1cn2nc(*)sc2n1] | 1 | 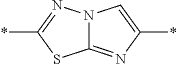 [SMILES: *c1cn2nc(*)sc2n1] | Ph | Ph |
| 57 | 1 | 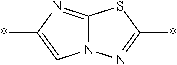 [SMILES: *c1cn2nc(*)sc2n1] | 1 | 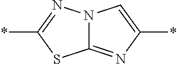 [SMILES: *c1cn2nc(*)sc2n1] | 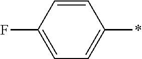 [SMILES: *c1ccc(F)cc1] | 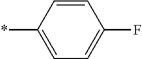 [SMILES: *c1ccc(F)cc1] |
| 58 | 1 | 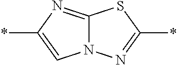 [SMILES: *c1cn2nc(*)sc2n1] | 1 | 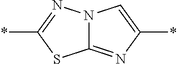 [SMILES: *c1cn2nc(*)sc2n1] | 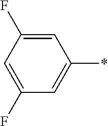 [SMILES: *c1cc(F)cc(F)c1] | 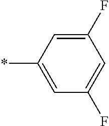 [SMILES: *c1cc(F)cc(F)c1] |
| 59 | 1 | 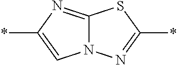 [SMILES: *c1cn2nc(*)sc2n1] | 1 | 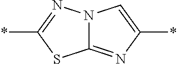 [SMILES: *c1cn2nc(*)sc2n1] | 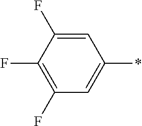 [SMILES: *c1cc(F)c(F)c(F)c1] | 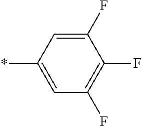 [SMILES: *c1cc(F)c(F)c(F)c1] |
| 80 | 1 | 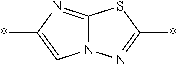 [SMILES: *c1cn2nc(*)sc2n1] | 1 | 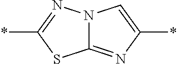 [SMILES: *c1cn2nc(*)sc2n1] | 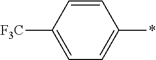 [SMILES: *c1ccc(C(F)(F)F)cc1] | 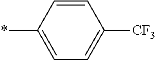 [SMILES: *c1ccc(C(F)(F)F)cc1] |
| 61 | 1 | 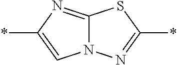 [SMILES: *c1cn2nc(*)sc2n1] | 1 | 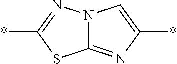 [SMILES: *c1cn2nc(*)sc2n1] | 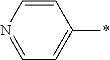 [SMILES: *c1ccncc1] | 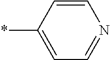 [SMILES: *c1ccncc1] |
| 62 | 1 | 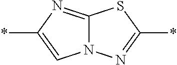 [SMILES: *c1cn2nc(*)sc2n1] | 1 | 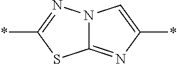 [SMILES: *c1cn2nc(*)sc2n1] | 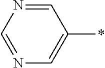 [SMILES: *c1cncnc1] | 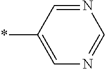 [SMILES: *c1cncnc1] |
| 63 | 1 | 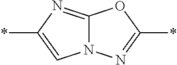 [SMILES: *c1cn2nc(*)oc2n1] | 1 | 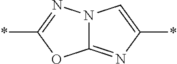 [SMILES: *c1cn2nc(*)oc2n1] | Ph | Ph |
| 64 | 1 | 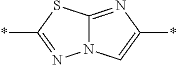 [SMILES: *c1cn2nc(*)sc2n1] | 1 | 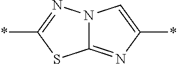 [SMILES: *c1cn2nc(*)sc2n1] | Ph | Ph |
| 85 | 1 | 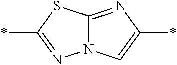 [SMILES: *c1cn2nc(*)sc2n1] | 1 | 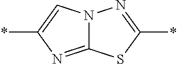 [SMILES: *c1cn2nc(*)sc2n1] | Ph | Ph |

A molecular weight of the specific compound is not particularly limited, but is preferably 550 to 1200, and more preferably 600 to 900. In a case where the molecular weight is 1200 or less, a vapor deposition temperature is not increased, and the compound is not easily decomposed. In a case where the molecular weight is 550 or more, a glass transition point of a vapor deposition film is not lowered, and the heat resistance of the photoelectric conversion element is improved.

The specific compound is particularly useful as a material of the photoelectric conversion film used for the imaging element, the optical sensor, or a photoelectric cell. The specific compound can also be used as a coloring material, a liquid crystal material, an organic semiconductor material, a charge transport material, a pharmaceutical material, and a fluorescent diagnostic material.

The specific compound is preferably a compound in which an ionization potential in a single film is −5.0 to −6.0 eV from the viewpoints of matching of energy levels between the compound and the n-type semiconductor material described later.

The maximum absorption wavelength of the specific compound is not particularly limited and is, for example, preferably within a wavelength range of 350 to 550 nm and more preferably within a wavelength range of 400 to 550 nm.

The maximum absorption wavelength is a value measured in a solution state (solvent: chloroform) by an absorption spectrum of the specific compound being adjusted to a concentration having an absorbance of about 0.5 to 1. However, in a case where the specific compound is not soluble in chloroform, a value measured by using the specific compound in which the specific compound is vapor-deposited and formed into a film state is defined as a maximum absorption wavelength of the specific compound.

The maximum absorption wavelength of the photoelectric conversion film is not particularly limited and is, for example, preferably within a wavelength range of 300 to 700 nm and more preferably within a wavelength range of 400 to 700 nm.

From the viewpoint of the responsiveness of the photoelectric conversion element, a content of the specific compound in the photoelectric conversion film (=film thickness of specific compound in terms of single layer/film thickness of photoelectric conversion film×100) is preferably 15% to 75% by volume, more preferably 20% to 60% by volume, and still more preferably 25% to 40% by volume.

The specific compound may be used alone, or two or more thereof may be used in combination.

### <Coloring Agent>

The photoelectric conversion film preferably contains a coloring agent as another component in addition to the specific compound described above.

The coloring agent is preferably an organic coloring agent.

Examples of the coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a quinoxaline coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, compounds disclosed in paragraphs [0083] to [0089] of JP2014-82483A, compounds disclosed in paragraphs [0029] to [0033] of JP2009-167348A, compounds disclosed in paragraphs [0197] to [0227] of JP2012-77064A, compounds disclosed in paragraphs [0035] to [0038] of WO2018-105269A, compounds disclosed in paragraphs [0041] to [0043] of WO2018-186389A, compounds disclosed in paragraphs [0059] to [0062] of WO2018-186397A, compounds disclosed in paragraphs [0078] to [0083] of WO2019-009249A, compounds disclosed in paragraphs [0054] to [0056] of WO2019-049946A, compounds disclosed in paragraphs [0059] to [0063] of WO2019-054327A, compounds disclosed in paragraphs [0086] and [0087] of WO2019-098161A, and compounds disclosed in paragraphs [0085] to [0114] of WO2020-013246.

A content of the coloring agent with respect to the total content of the specific compound and the coloring agent in the photoelectric conversion film (=(film thickness of coloring agent in terms of single layer/(film thickness of specific compound in terms of single layer+film thickness of coloring agent in terms of single layer)×100)) is preferably 15% to 75% by volume, more preferably 20% to 60% by volume, and still more preferably 25% to 50% by volume.

The coloring agent may be used alone, or two or more thereof may be used in combination.

<n-Type Semiconductor Material>

The photoelectric conversion film preferably further includes the n-type semiconductor material as another component in addition to the specific compound and coloring agent described above.

The n-type semiconductor material is an acceptor-property organic semiconductor material (compound), and refers to an organic compound having a property of easily accepting an electron.

More specifically, the n-type semiconductor material is preferably an organic compound having a higher electron affinity than that of the specific compound in a case where the n-type semiconductor material is used by being brought in contact with the above-described specific compound.

In the present specification, a value (value multiplied by −1) of a reciprocal number of the LUMO value obtained by the calculation of B3LYP/6-31G (d) using Gaussian '09 (software manufactured by Gaussian, Inc.) as a value of the electron affinity.

In addition, the n-type semiconductor material is preferably an organic compound having a higher electron affinity than the coloring agent in a case where the n-type semiconductor material is used by being brought in contact with the above-described coloring agent.

The electron affinity of the n-type semiconductor material is preferably 3.0 to 5.0 eV.

Examples of the n-type semiconductor material include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one of a nitrogen atom, an oxygen atom, or a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative; oxadiazole derivative; anthraquinodimethane derivatives; diphenylquinone derivatives; bathocuproine, bathophenanthroline, and derivatives thereof; triazole compounds; a distyrylarylene derivative; a metal complex having a nitrogen-containing heterocyclic compound as a ligand; a silole compound; and compounds disclosed in paragraphs [0056] and [0057] of JP2006-100767A.

Among these, it is preferable that the n-type semiconductor material includes fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. The fullerene derivative is preferably compounds described in JP2007-123707A.

In a case where the photoelectric conversion film includes the n-type semiconductor material, a content of the n-type semiconductor material with respect to the total content of the specific compound, the coloring agent, and the n-type semiconductor material (=(film thicknesses of n-type semiconductor material in terms of single layer/(film thickness of specific compound in terms of single layer+film thicknesses of coloring agent in terms of single layer+film thickness of n-type semiconductor material in terms of single layer)×100)) is preferably 15% to 75% by volume, more preferably 20% to 60% by volume, and still more preferably 25% to 50% by volume.

The n-type semiconductor material may be used alone, or two or more thereof may be used in combination.

In addition, in a case where the n-type semiconductor material includes fullerenes, a content of the fullerenes with respect to a total content of the n-type semiconductor material (=(film thickness of fullerenes in terms of single layer/total film thickness of n-type semiconductor materials in terms of single layer)×100) is preferably 50% to 100% by volume, and more preferably 80% to 100% by volume.

The fullerenes may be used alone, or two or more thereof may be used in combination.

The molecular weight of the n-type semiconductor material is preferably 200 to 1200, and more preferably 200 to 1000.

The photoelectric conversion film is substantially preferably composed of the specific compound, the coloring agent, and the n-type semiconductor material. “The photoelectric conversion film is substantially composed of only the specific compound, the coloring agent, and the n-type semiconductor material” means “the total content of the specific compound, the coloring agent, and the n-type semiconductor material with respect to the total mass of the photoelectric conversion film is 95% to 100% by mass”.

In a case where the photoelectric conversion film contains a coloring agent, the photoelectric conversion film is preferably a mixture layer formed in a state where the specific compound and the coloring agent are mixed.

In addition, in a case where the photoelectric conversion film contains an n-type semiconductor material, the photoelectric conversion film is preferably a mixture layer formed in a state in which the specific compound, and the n-type semiconductor material are mixed.

In a case where the photoelectric conversion film contains a coloring agent and an n-type semiconductor material, the photoelectric conversion film is preferably a mixture layer formed in a state in which the specific compound, the coloring agent, and the n-type semiconductor material are mixed.

The mixture layer is a layer in which two or more materials are mixed in a single layer.

The photoelectric conversion film containing the specific compound is a non-light emitting film, and has a feature different from organic light emitting diodes (OLEDs). The non-light emitting film is intended for a film having a light emission quantum efficiency of 1% or less, and the light emission quantum efficiency is preferably 0.5% or less, and more preferably 0.1% or less.

### <Film Formation Method>

The photoelectric conversion film can be formed mostly by a dry film formation method. Examples of the dry film formation method include a physical vapor deposition method such as a vapor deposition method (in particular, a vacuum vapor deposition method), a sputtering method, an ion plating method, and a molecular beam epitaxy (MBE) method, and a chemical vapor deposition (CVD) method such as plasma polymerization. Among these, the vacuum vapor deposition method is preferable. In a case where the photoelectric conversion film is formed by the vacuum vapor deposition method, manufacturing conditions such as a degree of vacuum and a vapor deposition temperature can be set according to the normal method.

The thickness of the photoelectric conversion film is preferably 10 to 1000 nm, more preferably 50 to 800 nm, still more preferably 50 to 500 nm, and particularly preferably 50 to 400 nm.

### [Electrode (Conductive Film)]

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO), fluorine doped tin oxide (FTO)) doped with antimony, fluorine, or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; organic conductive materials such as polyaniline, polythiophene, and polypyrrole; carbon materials such as graphene and carbon nanotubes. Among these, conductive metal oxides are preferable from the viewpoints of high conductivity, transparency, and the like.

In general, in a case where the conductive film is made to be thinner than a certain range, a resistance value is rapidly increased. However, in the solid-state imaging element into which the photoelectric conversion element according to the present embodiment is incorporated, the sheet resistance may be, for example, 100 to 10000Ω/□, and a degree of freedom of a range of the film thickness that can be thinned is large. In addition, as the thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the film thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, and conductive compounds (for example, titanium nitride (TiN)) such as oxides or nitrides of these metals; mixtures or laminates of these metals and conductive metal oxides; organic conductive materials such as polyaniline, polythiophene, and polypyrrole; carbon materials such as graphene and carbon nanotubes.

The method of forming electrodes is not particularly limited, and can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

### [Charge Blocking Film: Electron Blocking Film, Hole Blocking Film]

It is also preferable that the photoelectric conversion element according to the embodiment of the present invention has one or more interlayers between the conductive film and the transparent conductive film, in addition to the photoelectric conversion film. An example of the interlayer includes a charge blocking film. In a case where the photoelectric conversion element has this film, the characteristics (such as photoelectric conversion efficiency and responsiveness) of the obtained photoelectric conversion element are more excellent. Examples of the charge blocking film include an electron blocking film and a hole blocking film. Hereinafter, each of the films will be described in detail.

### <Electron Blocking Film>

The electron blocking film is a donor organic semiconductor material (compound), and a p-type organic semiconductor described below can be used, for example. The p-type organic semiconductor may be used alone, or two or more thereof may be used in combination.

Examples of the p-type organic semiconductor include triarylamine compounds (for example, N, N′-bis (3-methylphenyl)-(1,1′-biphenyl)-4,4′-diamine (TPD), 4,4′-bis [N-(naphthyl)-N-phenyl-amino] biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in [0044] to [0051] of JP2015-153910A, and compounds disclosed in paragraphs [0086] to [0090] of JP2012-94660A, pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1] benzothieno [3,2-b] thiophene (BTBT) derivative, a thieno [3,2-f: 4,5-f] bis [1] benzothiophene (TBBT) derivative, compounds disclosed in paragraphs [0031] to [0036] of JP2018-14474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-54228A, compounds disclosed in paragraphs [0045] to [0055] of WO2019-058995A, compounds disclosed in paragraphs [0063] to [0089] of WO2019-081416A, compounds disclosed in paragraphs [0033] to [0036] of JP2019-80052A, compounds disclosed in paragraphs [0044] to [0054] of WO2019-054125A, compounds disclosed in paragraphs [0041] to [0046] of WO2019-093188A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, and a fluoranthene derivative), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

Examples of the p-type organic semiconductor include compounds having an ionization potential smaller than that of the n-type semiconductor material, and in a case where this condition is satisfied, the above-described coloring agents can be also used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### <Hole Blocking Film>

A hole blocking film is an acceptor-property organic semiconductor material (compound), and the n-type semiconductor material described above and the like can be used.

The method of manufacturing the charge blocking film is not particularly limited, and examples thereof include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each thickness of the charge blocking films (the electron blocking film and the hole blocking film) is preferably 3 to 200 nm, more preferably 5 to 100 nm, and still more preferably 5 to 30 nm.

### [Substrate]

The photoelectric conversion element may further include a substrate. Types of the substrate to be used are not particularly limited, and examples of the substrate include a semiconductor substrate, a glass substrate, and a plastic substrate.

A position of the substrate is not particularly limited, and in general, the conductive film, the photoelectric conversion film, and the transparent conductive film are laminated on the substrate in this order.

### [Sealing Layer]

The photoelectric conversion element may further include a sealing layer. The performance of a photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, or metal nitride, and metal nitride oxide which are dense and into which water molecules do not permeate.

The material of the sealing layer may be selected and the sealing layer may be manufactured according to the description in paragraphs [0210] to [0215] of JP2011-082508A.

### [Imaging Element and Optical Sensor]

An example of the application of the photoelectric conversion element includes an imaging element. The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

The imaging element is mounted on an imaging element such as a digital camera and a digital video camera, an electronic endoscope, and imaging modules such as a cellular phone.

The photoelectric conversion element according to the embodiment of the present invention is also preferably used for an optical sensor including the photoelectric conversion element according to the embodiment of the present invention. The photoelectric conversion element may be used alone as the optical sensor, and the photoelectric conversion element may be used as a line sensor in which the photoelectric conversion elements are linearly arranged or as a two-dimensional sensor in which the photoelectric conversion elements are arranged in a plane shape.

### [Compound]

The present invention also relates to a compound.

The compound according to the embodiment of the present invention is the same compound as the above-described specific compound (compound represented by Formula (1)), and preferred conditions are also the same.

### EXAMPLES

The present invention will be described in more detail based on Examples below. Materials, used amounts, ratios, treatment contents, treatment procedures, and the like described in the following Examples can be appropriately changed within the range that does not depart from the gist of the present invention. Therefore, the range of the present invention should not be limitatively interpreted by the following Examples.

### [Compound (Evaluation Compound)]

### <Synthesis of Compound (1-1)>

A compound (1-1) that is a specific compound was synthesized according to the following scheme.

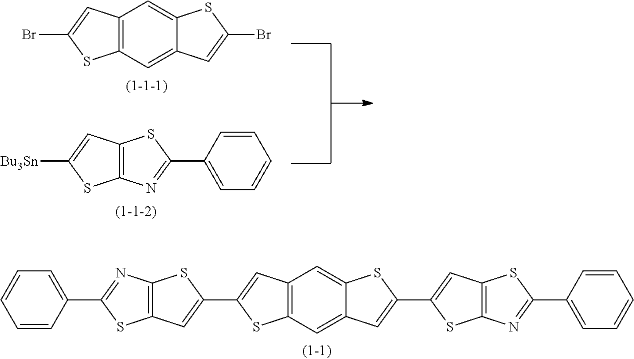

A compound (1-1-1) (1 mmol), a compound (1-1-2) (2.4 mmol), XPhos Pd G3 (0.03 mmol), and 16 mL of 4-methyltetrahydropyrane were added to a glass reaction container to obtain a mixed solution. After the inside of the reaction container was replaced with nitrogen, the mixed solution was reacted at 100° C. for 5 hours. The mixed solution was left to cool to room temperature (25° C.), and precipitates precipitated in the mixed solution were then collected by filtration. A solid (filter product) thus obtained was suspended in chlorobenzene, heated at 140° C. for one hour, and collected by filtration. A solid (filter product) thus obtained was dried under reduced pressure and then sublimated and purified to obtain 0.4 mmol of the compound (1-1).

The measurement result of the obtained compound (1-1) by LDI-MS (soft laser desorption ionization mass analysis) was as follows.

LDI-MS: 620.9 (M⁺+H)

XPhos Pd G3 is (2-dicyclohexylphosphino-2′,4′,6′-triisopropyl-1,1′-biphenyl)[2-(2′-amino-1,1′-biphenyl)]palladium (II) methanesulfonate.

### <Synthesis of Compound (1-24)>

A compound (1-24) that is the specific compound was synthesized according to the following scheme.

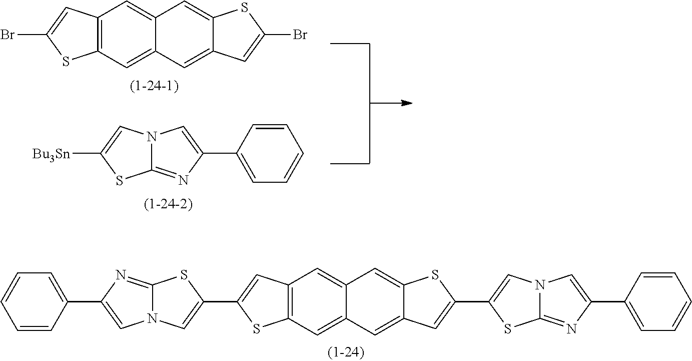

A compound (1-24-1) (1 mmol), a compound (1-24-2) (2.4 mmol), XPhos Pd G3 (0.03 mmol), and 4-methyltetrahydropyrane (16 mL) were added to a glass reaction container to obtain a mixed solution. After the inside of the reaction container was replaced with nitrogen, the mixed solution was reacted at 100° C. for 5 hours. The mixed solution was left to cool to room temperature (25° C.), and precipitates precipitated in the mixed solution were then collected by filtration. A solid (filter product) thus obtained was suspended in chlorobenzene, heated at 140° C. for one hour, and collected by filtration. A solid (filter product) thus obtained was dried under reduced pressure and then sublimated and purified to obtain 0.5 mmol of the compound (1-24).

The measurement result of the obtained compound (1-24) by LDI-MS was as follows.

LDI-MS: 636.9 (M⁺+H)

Other specific compounds were also synthesized with reference to the above-described synthesis method.

The specific compound and comparative compound used in a test are illustrated below.

Hereinbelow, compounds (1-1) to (1-27) are specific compounds.

Hereinafter, the specific compound and the Comparative compound are collectively referred to as an evaluation compound.

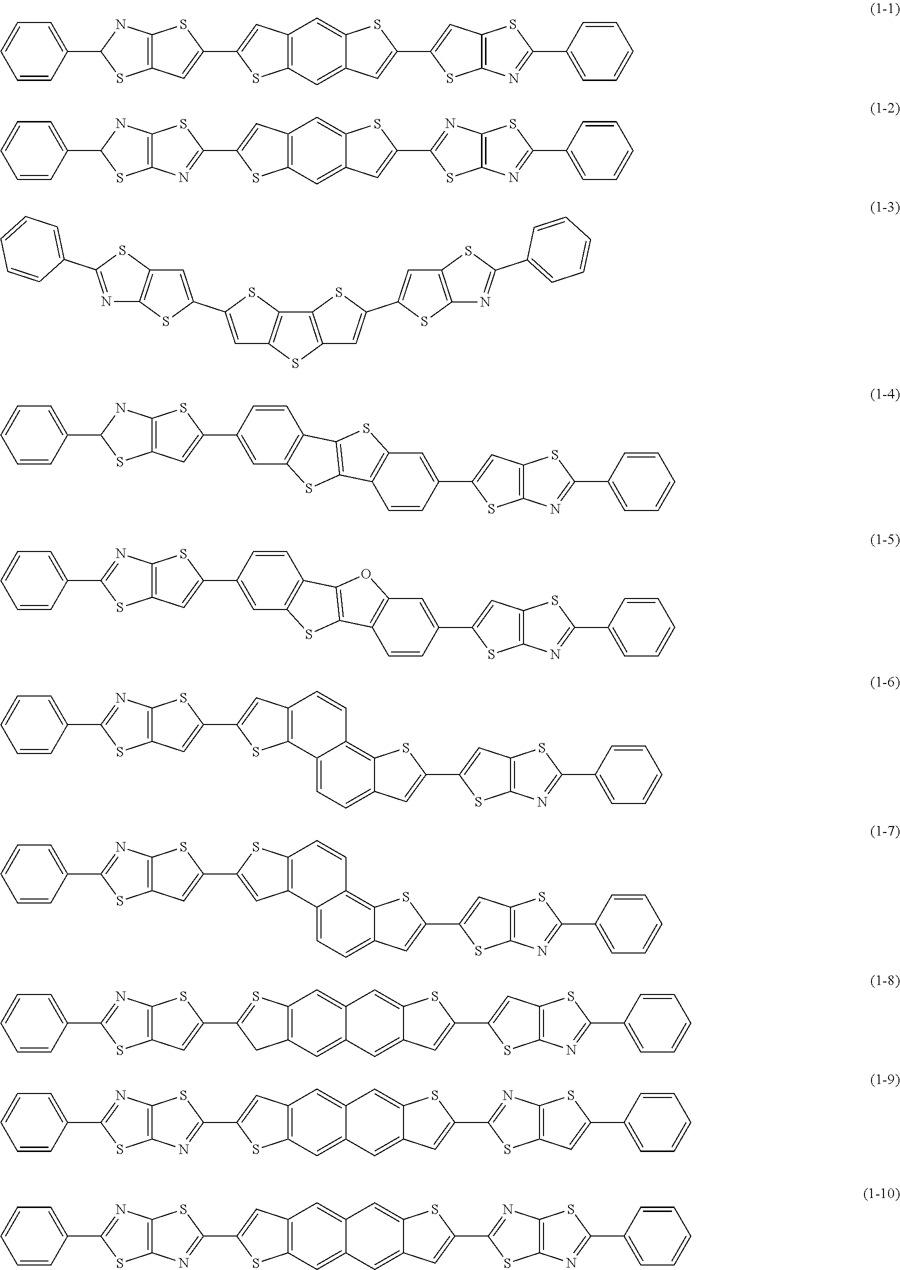

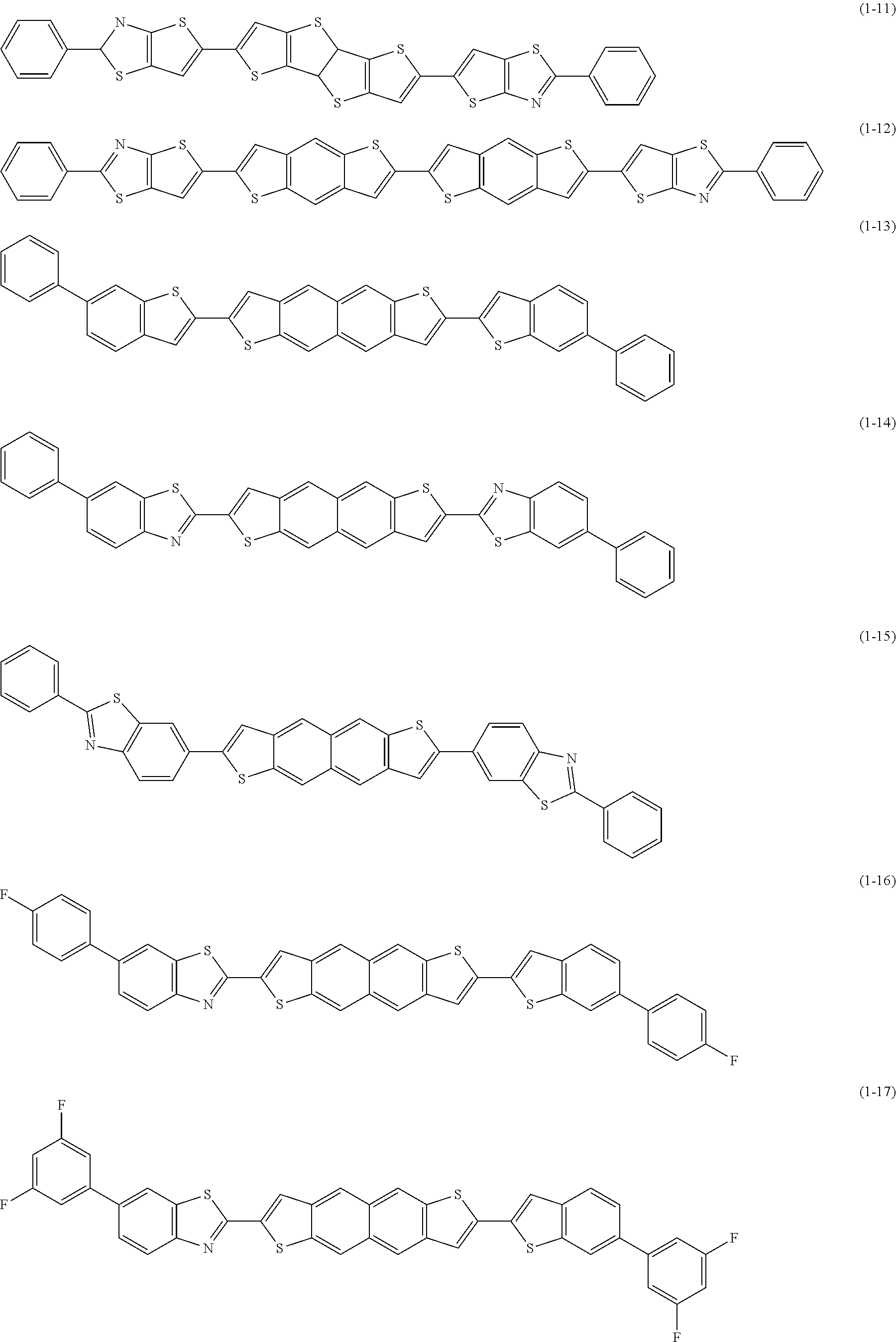

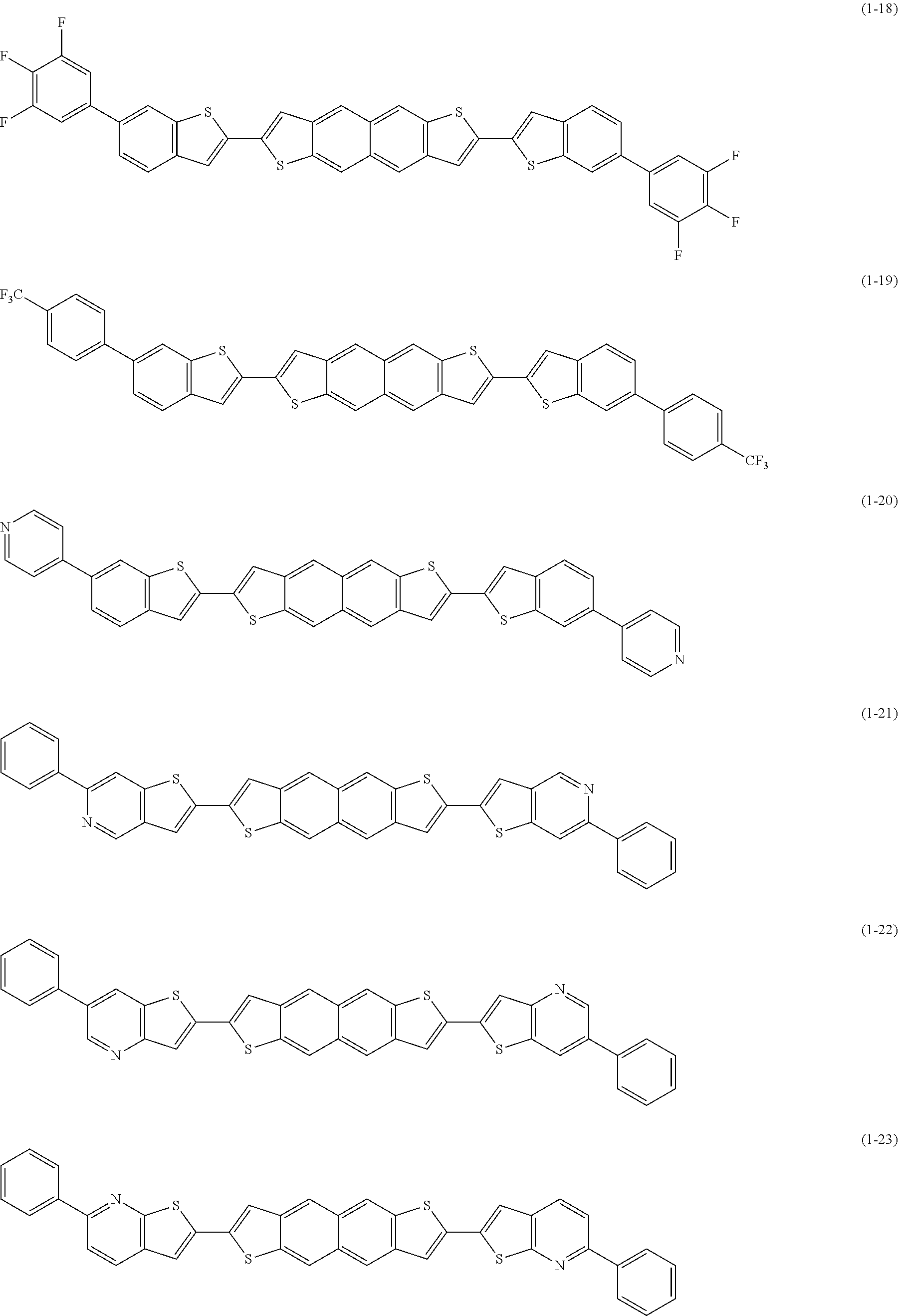

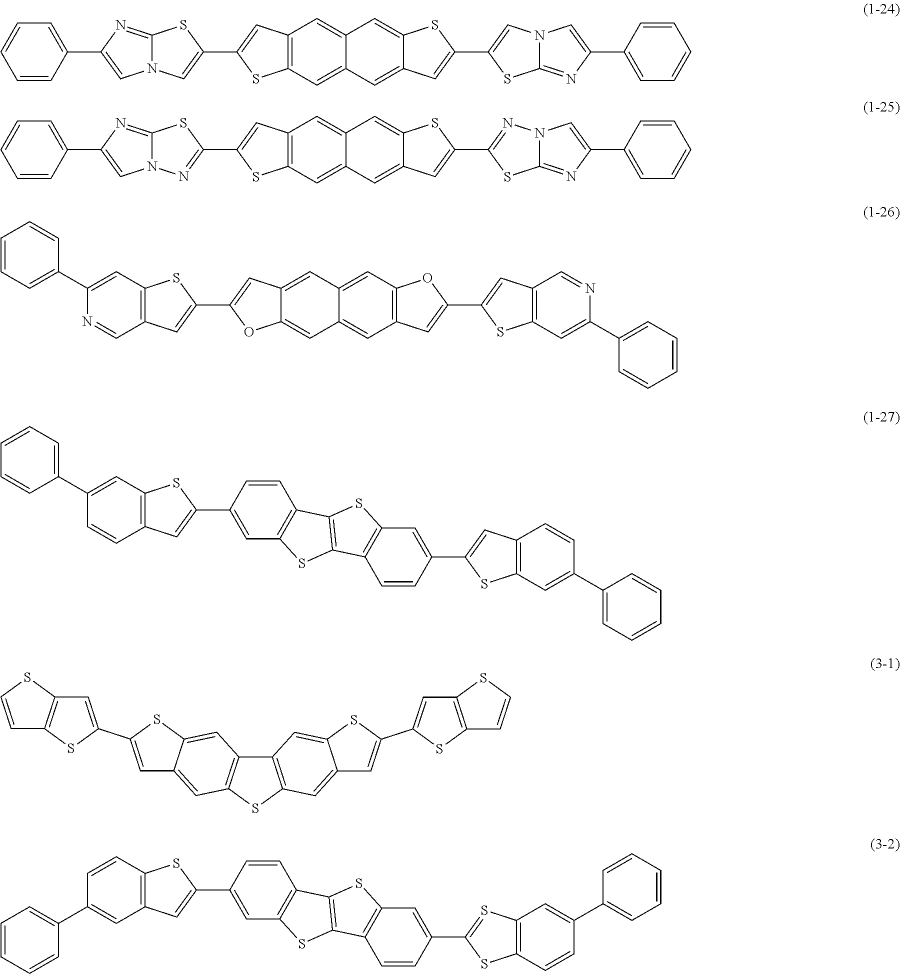

### [Coloring Agent (Coloring Agent for Evaluation)]

The coloring agents illustrated below were coloring agents used in the evaluation in Examples, and were used in the production of photoelectric conversion elements described later.

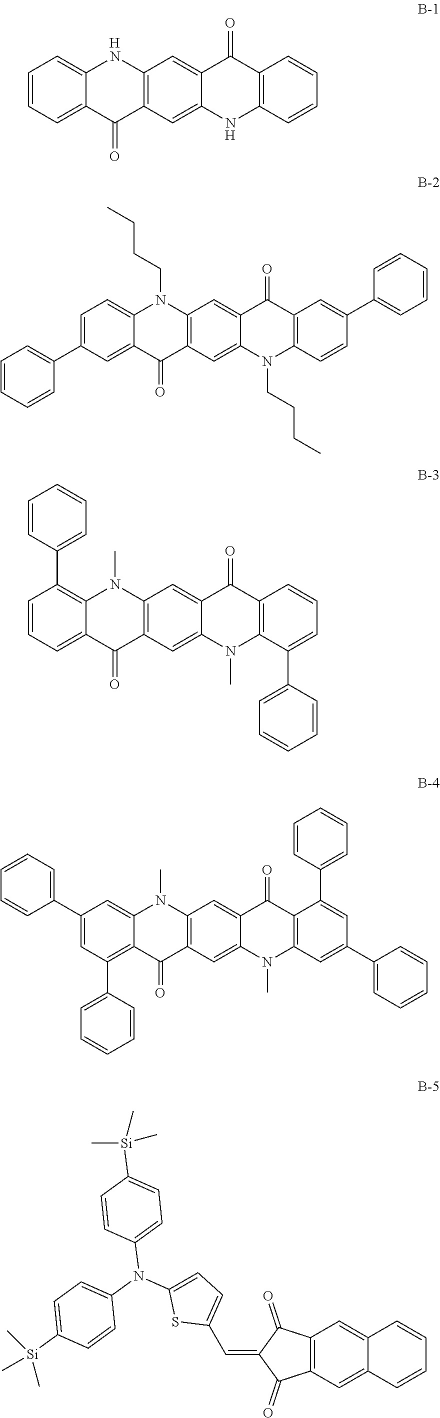

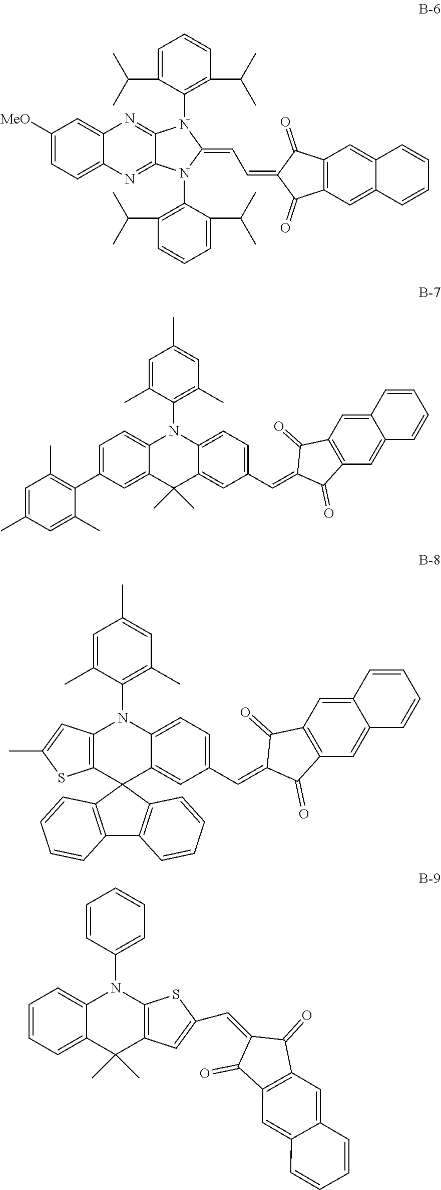 [SMILES: COc1ccc2nc3c(nc2c1)N(c1c(C(C)C)cccc1C(C)C)/C(=C/C=C1C(=O)c2cc4ccccc4cc2C1=O)N3c1c(C(C)C)cccc1C(C)C ; Cc1cc(C)c(-c2ccc3c(c2)C(C)(C)c2cc(C=C4C(=O)c5cc6ccccc6cc5C4=O)ccc2N3c2c(C)cc(C)cc2C)c(C)c1 ; Cc1cc(C)c(N2c3ccc(C=C4C(=O)c5cc6ccccc6cc5C4=O)cc3C3(c4ccccc4-c4ccccc43)c3sc(C)cc32)c(C)c1 ; CC1(C)c2ccccc2N(c2ccccc2)c2sc(C=C3C(=O)c4cc5ccccc5cc4C3=O)cc21]

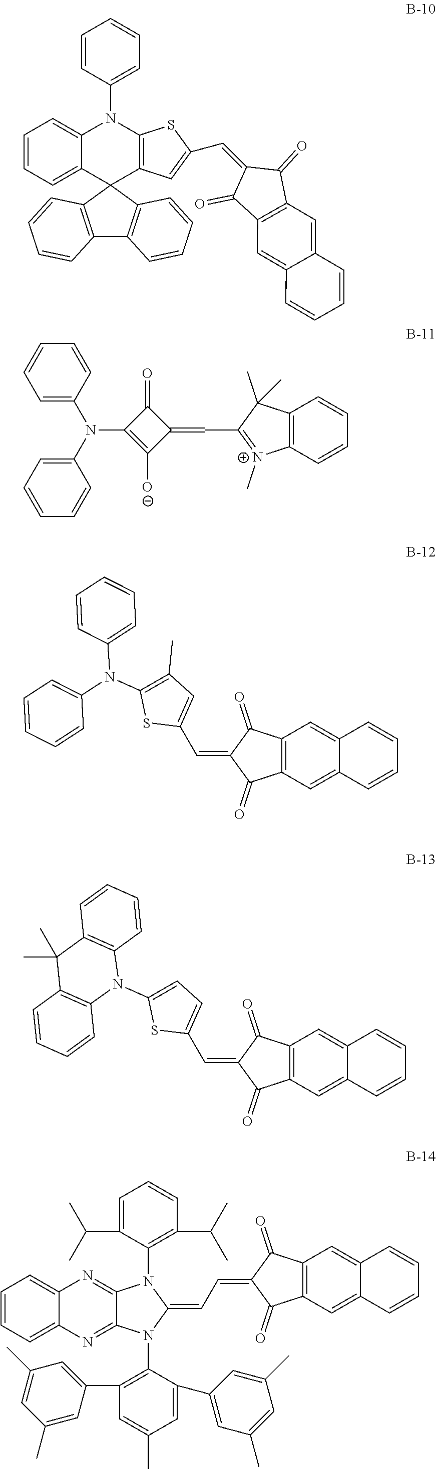 [SMILES: O=C1C(=Cc2cc3c(s2)N(c2ccccc2)c2ccccc2C32c3ccccc3-c3ccccc32)C(=O)c2cc3ccccc3cc21 ; C[N+]1=C(C=C2C(=O)C(N(c3ccccc3)c3ccccc3)=C2[O-])C(C)(C)c2ccccc21 ; Cc1cc(C=C2C(=O)c3cc4ccccc4cc3C2=O)sc1N(c1ccccc1)c1ccccc1 ; CC1(C)c2ccccc2N(c2ccc(C=C3C(=O)c4cc5ccccc5cc4C3=O)s2)c2ccccc21 ; Cc1cc(C)cc(-c2cc(C)cc(-c3cc(C)cc(C)c3)c2N2/C(=C/C=C3C(=O)c4cc5ccccc5cc4C3=O)N(c3c(C(C)C)cccc3C(C)C)c3nc4ccccc4nc32)c1]

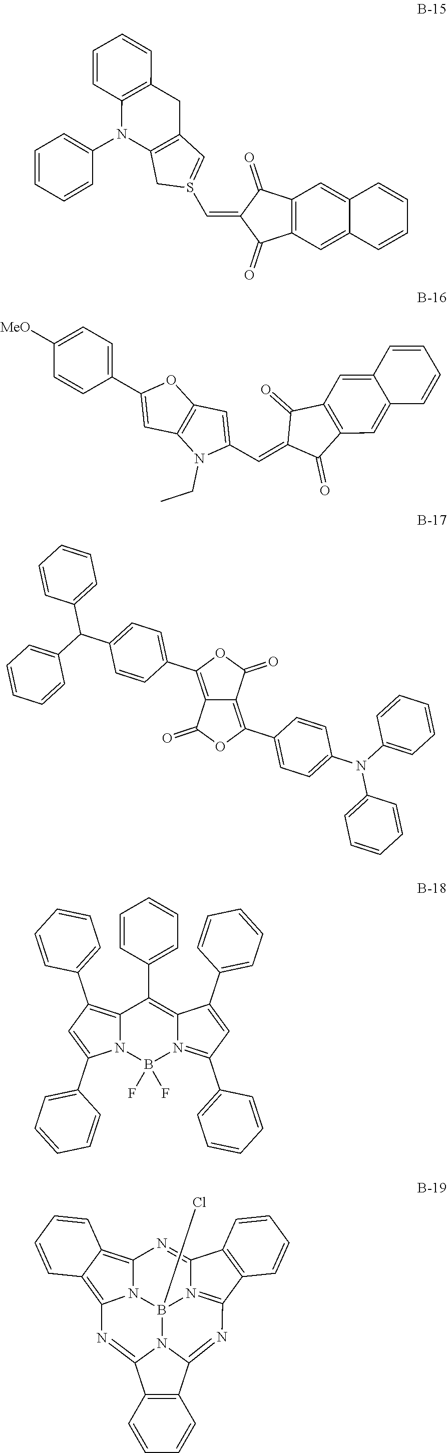

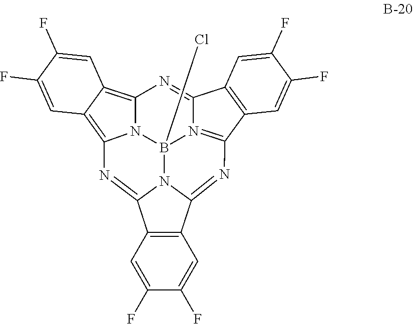

[n-Type Semiconductor Material]

Fullerene C₆₀ was used for the production of photoelectric conversion elements described later, as a n-type semiconductor material used for evaluations.

### [Test]

### <Examples and Comparative Examples: Production of Photoelectric Conversion Element>

The photoelectric conversion element of the form illustrated in FIG. 2 was produced using the obtained compounds. Here, the photoelectric conversion element includes a lower electrode 11, an electron blocking film 16A, a photoelectric conversion film 12, a hole blocking film 16B, and an upper electrode 15.

Specifically, an amorphous ITO was formed into a film on a glass substrate by a sputtering method to form the lower electrode 11 (thickness: 30 nm). Furthermore, a compound (C-1) described below was formed into a film on the lower electrode 11 by a vacuum thermal vapor deposition method to form the electron blocking film 16A (thickness: 30 nm). Each of the evaluation compounds illustrated in Table 1, the n-type semiconductor material, and the coloring agent were co-vapor-deposited on the electron blocking film 16A to form the photoelectric conversion film 12 as a mixture layer. Ratios of vapor deposition rates of the evaluation compound, the n-type semiconductor material, and the coloring agent were adjusted so that a film thickness of each of these components in the photoelectric conversion film in terms of a single layer is each of ratios illustrated in the “Component ratio” column of Table 1.

Furthermore, a compound (C-2) described below was vapor-deposited on the photoelectric conversion film 12 to form the hole blocking film 16B (thickness: 10 nm). Amorphous ITO was formed into a film on the hole blocking film 16B by a sputtering method to form the upper electrode 15 (the transparent conductive film) (thickness: 10 nm). A SiO film was formed as a sealing layer on the upper electrode 15 by a vacuum vapor deposition method, and thereafter, an aluminum oxide (Al₂O₃) layer was formed thereon by an atomic layer chemical vapor deposition (ALCVD) method to produce a photoelectric conversion element obtained in each of Examples or Comparative Examples.

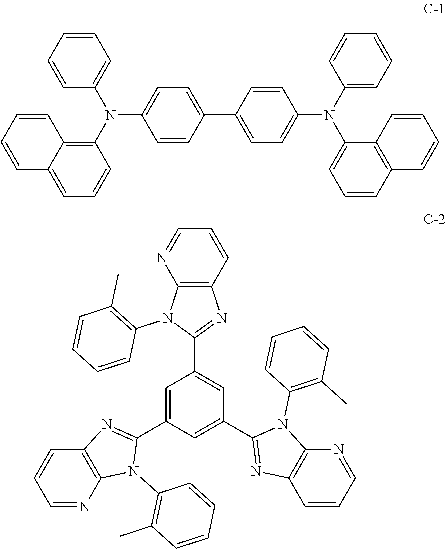 [SMILES: c1ccc(N(c2ccc(-c3ccc(N(c4ccccc4)c4cccc5ccccc45)cc3)cc2)c2cccc3ccccc23)cc1 ; Cc1ccccc1-n1c(-c2cc(-c3nc4cccnc4n3-c3ccccc3C)cc(-c3nc4cccnc4n3-c3ccccc3C)c2)nc2cccnc21]

### <Evaluation of Dark Current>

The dark current of each of the obtained photoelectric conversion elements was measured by the following method.

A voltage was applied to the lower electrode and the upper electrode of each of the photoelectric conversion elements to have an electric field strength of 2.5×10⁵ V/cm and current values (dark current) in a dark place were measured. As a result, it was confirmed that all of the photoelectric conversion elements had a dark current of 50 nA/cm² or less, which indicates that all of the photoelectric conversion elements had a sufficiently low dark current.

### <Evaluation of Photoelectric Conversion Efficiency (Quantum Efficiency)>

The drive of each photoelectric conversion element thus obtained was confirmed by a method below.

A voltage was applied to each photoelectric conversion element to have an electric field strength of 2.0×10⁵ V/cm. Thereafter, light was emitted from the upper electrode (transparent conductive film) side to evaluate the photoelectric conversion efficiency (external quantum efficiency) within the visible light range (light having a wavelength of 400 to 700 nm).

An integral value of the photoelectric conversion efficiency in light having a wavelength of 400 to 550 nm was used to calculate a relative ratio of the integral value of the photoelectric conversion efficiency by Expression (S), and the evaluation according to the following standard was carried out.

As the relative ratio is increased, the photoelectric conversion efficiency (particularly, the photoelectric conversion efficiency with respect to light having a wavelength of 400 to 550 nm) is more excellent and preferable.

Relative ratio=(Integral value of the photoelectric conversion efficiency of the photoelectric conversion element to be evaluated with light having a wavelength of 400 to 550 nm)/(Integral value of the photoelectric conversion efficiency of the photoelectric conversion element of Example 1-1 with light having a wavelength of 400 to 550 nm) Expression (S):

A: The relative ratio of the integral values of the photoelectric conversion efficiency is 1.4 or more.

B: The relative ratio of the integral values of the photoelectric conversion efficiency is 1.2 or more and less than 1.4.

C: The relative ratio of the integral values of the photoelectric conversion efficiency is 1.0 or more and less than 1.2.

D: The relative ratio of the integral values of the photoelectric conversion efficiency is 0.8 or more and less than 1.0.

E: The relative ratio of the integral values of the photoelectric conversion efficiency is less than 0.8.

### <Evaluation of Electric Field Strength Dependence of Photoelectric Conversion Efficiency>

The electric field strength dependence of the quantum efficiency in each of the obtained photoelectric conversion elements was confirmed by the following method.

A voltage was applied to each photoelectric conversion element to have an electric field strength of 1.5×10⁵ V/cm. Thereafter, light was emitted from the upper electrode (transparent conductive film) side to evaluate the photoelectric conversion efficiency (external quantum efficiency) within the visible light range (light having a wavelength of 400 to 700 nm).

A voltage was further applied to each photoelectric conversion element to have an electric field strength of 2.0×10⁵ V/cm. Thereafter, light was emitted from the upper electrode (transparent conductive film) side to evaluate the photoelectric conversion efficiency (external quantum efficiency) within the visible light range (light having a wavelength of 400 to 700 nm).

The integral value of the photoelectric conversion efficiency measured at each electric field strength in light having a wavelength of 400 to 550 nm was used to calculate the photoelectric conversion efficiency ratio by the following Expression and evaluate the electric field strength dependence of the photoelectric conversion efficiency according to the following standard.

As the photoelectric conversion efficiency ratio is closer to 1, the electric field strength dependence of the photoelectric conversion efficiency is small, which is preferable.

Photoelectric conversion efficiency ratio=(Integral value of the photoelectric conversion efficiency with light having a wavelength of 400 to 550 nm under a condition in which a voltage is applied to the photoelectric conversion element to be evaluated so that the electric field strength is 1.5×10⁵ V/cm)/(Integral value of the photoelectric conversion efficiency with light having a wavelength of 400 to 550 nm under a condition in which a voltage is applied to the photoelectric conversion element to be evaluated so that the electric field strength is 2.0×10⁵ V/cm)

A: The photoelectric conversion efficiency ratio is 0.9 or more and 1.0 or less.

B: The photoelectric conversion efficiency ratio is 0.8 or more and less than 0.9.

C: The photoelectric conversion efficiency ratio is 0.7 or more and less than 0.8.

D: The photoelectric conversion efficiency ratio is less than 0.7.

The features of the photoelectric conversion element of each of Examples and Comparative Examples and the results of tests conducted using the photoelectric conversion element of each Examples and Comparative Examples are illustrated in Table 1 below.

In Tables, the “Type” column in each of the “Evaluation compound” column, the “n-type semiconductor material” column, and the “Coloring agent” column indicates types of the components used in the production of the photoelectric conversion element.

The “Ar11” column indicates which of the groups represented by Formula (A1) to Formula (A6) is represented by the group represented by Ar¹¹ in the specific compound used.

TABLE 5

| | | | | | | Result | |
|---|---|---|---|---|---|---|---|
| | | | | | | | Electric field |
| | | | | | | | strength |
| | | | n-type | | | | dependence of |
| | Evaluation | | semiconductor | Coloring | | Photoelectric | photoelectric |
| | compound | | material | agent | Component | conversion | conversion |
| | Type | Ar11 | Type | Type | ratio | efficiency | efficiency |
| Example 1-1 | 1-1 | A1 | Fullerene | B-1 | 1:1:1 | C | C |
| Example 1-2 | 1-1 | A1 | Fullerene | B-6 | 1:1:1 | C | C |
| Example 1-3 | 1-1 | A1 | Fullerene | B-18 | 1:1:1 | C | C |
| Example 1-4 | 1-2 | A1 | Fullerene | B-2 | 1:1:1 | C | C |
| Example 1-5 | 1-3 | A2 | Fullerene | B-3 | 1:1:1 | C | C |
| Example 1-6 | 1-4 | A3 | Fullerene | B-4 | 1:1:1 | C | B |
| Example 1-7 | 1-5 | A3 | Fullerene | B-5 | 1:1:1 | C | B |
| Example 1-8 | 1-6 | A4 | Fullerene | B-6 | 1:1:1 | C | B |
| Example 1-9 | 1-7 | A4 | Fullerene | B-7 | 1:1:1 | C | B |
| Example 1-10 | 1-8 | A5 | Fullerene | B-8 | 1:1:1 | A | A |
| Example 1-11 | 1-9 | A5 | Fullerene | B-8 | 1:1:1 | A | A |
| Example 1-12 | 1-10 | A5 | Fullerene | B-9 | 1:1:1 | A | A |
| Example 1-13 | 1-11 | A6 | Fullerene | B-10 | 1:1:1 | C | B |
| Example 1-14 | 1-12 | A1 | Fullerene | B-11 | 1:1:1 | B | B |
| Example 1-15 | 1-13 | A5 | Fullerene | B-12 | 1:1:1 | B | A |
| Example 1-16 | 1-14 | A5 | Fullerene | B-12 | 1:1:1 | A | A |
| Example 1-17 | 1-15 | A5 | Fullerene | B-12 | 1:1:1 | B | A |
| Example 1-18 | 1-16 | A5 | Fullerene | B-13 | 1:1:1 | A | A |
| Example 1-19 | 1-17 | A5 | Fullerene | B-14 | 1:1:1 | A | A |
| Example 1-20 | 1-18 | A5 | Fullerene | B-15 | 1:1:1 | A | A |
| Example 1-21 | 1-19 | A5 | Fullerene | B-16 | 1:1:1 | A | A |
| Example 1-22 | 1-20 | A5 | Fullerene | B-17 | 1:1:1 | A | A |
| Example 1-23 | 1-21 | A5 | Fullerene | B-18 | 1:1:1 | A | A |
| Example 1-24 | 1-22 | A5 | Fullerene | B-18 | 1:1:1 | A | A |
| Example 1-25 | 1-23 | A5 | Fullerene | B-19 | 1:1:1 | A | A |
| Example 1-26 | 1-24 | A5 | Fullerene | B-20 | 1:1:1 | A | A |
| Example 1-27 | 1-25 | A5 | Fullerene | B-20 | 1:1:1 | A | A |
| Example 1-28 | 1-26 | A5 | Fullerene | B-18 | 1:1:1 | A | A |
| Example 1-29 | 1-27 | A3 | Fullerene | B-1 | 1:1:1 | C | B |
| Comparative | 3-1 | — | Fullerene | B-1 | 1:1:1 | E | D |
| Example 1-1 | | | | | | | |
| Comparative | 3-2 | — | Fullerene | B-1 | 1:1:1 | D | D |
| Example 1-2 | | | | | | | |

From the result illustrated in Table 1, it was confirmed that the photoelectric conversion element according to the embodiment of the present invention using the specific compound for the photoelectric conversion film was excellent to exhibit the effect of the present invention.

On the other hand, it has been confirmed that in a case where a compound different from the specific compound is used, the obtained photoelectric conversion element is inferior in the photoelectric conversion efficiency and also has a large electric field strength dependence of the photoelectric conversion efficiency.

In a case where the specific compound in which the group represented by Ar¹¹ is a group represented by Formula (A5) is used, the photoelectric conversion efficiency and/or the electric field strength dependence of the photoelectric conversion efficiency is more favorable. Therefore, it was confirmed that the effect of the present invention is more excellent in a case where the group represented by Ar¹¹ in the specific compound is a group represented by Formula (A5).

### Explanation of References

10a, 10b: photoelectric conversion element
11: conductive film (lower electrode)
12: photoelectric conversion film
15: transparent conductive film (upper electrode)
16A: electron blocking film
16B: hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 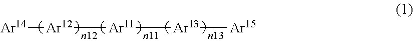
in Formula (1), n11 represents 1 or 2,
n12 and n13 each independently represent 0 or 1, where, at least one of n12 or n13 represents 1,
Ar¹¹ represents a fused polycyclic aromatic ring group consisting of a combination of one or more aromatic rings selected from the group consisting of a thiophene ring, a benzene ring, a furan ring, and a selenophene ring, where, the fused polycyclic aromatic ring group has three or four rings, the fused polycyclic aromatic ring group contains at least one thiophene ring, the fused polycyclic aromatic ring group may have a substituent,
Ar¹⁴ and Ar¹⁵ each independently represent an aryl group which may have a substituent or a heteroaryl group which may have a substituent, and
Ar¹² and Ar¹³ each independently represent any one of groups represented by Formula (2) to Formula (4), 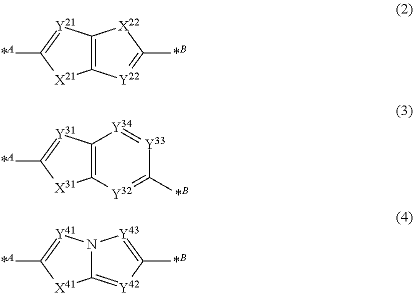
in Formula (2) to Formula (4), *^{A} and *^{B} each represent a bonding position,
in Formula (2), X²¹ and X²² each independently represent a sulfur atom, an oxygen atom, or a selenium atom, Y²¹ and Y²² each independently represent a nitrogen atom or —CR═, and R represents a hydrogen atom or a substituent, where, at least one of Y²¹ or Y²² represents a nitrogen atom,
in Formula (3), X³¹ represents a sulfur atom, an oxygen atom, or a selenium atom, Y³¹ to Y³⁴ each independently represent a nitrogen atom or —CR═, and R represents a hydrogen atom or a substituent,
in Formula (4), X⁴¹ represents a sulfur atom, an oxygen atom, or a selenium atom, Y⁴¹ to Y⁴³ each independently represent a nitrogen atom or —CR═, and R represents a hydrogen atom or a substituent.

2. The photoelectric conversion element according to claim 1,
wherein in Formula (1), a group represented by Ar¹¹ is any one of groups represented by Formula (A1) to Formula (A6), 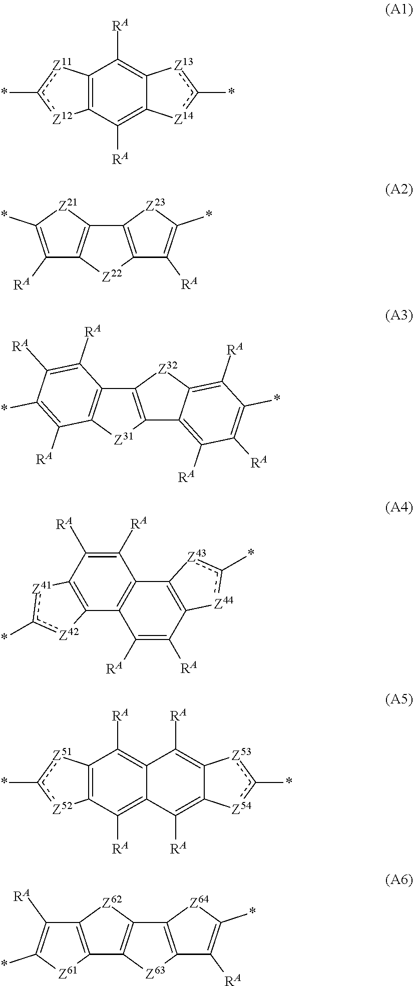
in Formulae (A1) to (A6), * represents a bonding position,
in Formula (A1), one of Z¹¹ or Z¹² represents a sulfur atom, and the other of Z¹¹ or Z¹² represents —CR═, one of Z¹³ or Z¹⁴ represents a sulfur atom, an oxygen atom, or a selenium atom, and the other of Z¹³ or Z¹⁴ represents —CR═, and R and R^{A} each independently represent a hydrogen atom or a substituent,
in Formula (A2), Z²¹ to Z²³ each independently represent a sulfur atom, an oxygen atom, or a selenium atom, at least one of Z²¹, Z²², or Z²³ represents a sulfur atom, and R^{A} represents a hydrogen atom or a substituent,
in Formula (A3), Z³¹ and Z³² each independently represent a sulfur atom, an oxygen atom, or a selenium atom, at least one of Z³¹ or Z³² represents a sulfur atom, and R^{A} represents a hydrogen atom or a substituent,
in Formula (A4), one of Z⁴¹ or Z⁴² represents a sulfur atom, and the other of Z⁴¹ or Z⁴² represents —CR═, one of Z⁴³ or Z⁴⁴ represents a sulfur atom, an oxygen atom, or a selenium atom, and the other of Z⁴³ or Z⁴⁴ represents —CR═, and R and R^{A} each independently represent a hydrogen atom or a substituent,
in Formula (A5), one of Z⁵¹ or Z⁵² represents a sulfur atom, and the other of Z⁵¹ or Z⁵² represents —CR═, one of Z⁵³ or Z⁵⁴ represents a sulfur atom, an oxygen atom, or a selenium atom, and the other of Z⁵³ or Z⁵⁴ represents —CR═, and R and R^{A} each independently represent a hydrogen atom or a substituent,
in Formula (A6), Z⁶¹ to Z⁶⁴ each independently represent a sulfur atom, an oxygen atom, or a selenium atom, at least one of Z⁶¹, Z⁶², Z⁶³, or Z⁶⁴ represents a sulfur atom, and R^{A} represents a hydrogen atom or a substituent.

3. The photoelectric conversion element according to claim 2,
wherein in Formula (A1), one of Z¹¹ or Z¹² represents a sulfur atom, and the other of Z¹¹ or Z¹² represents —CR═, and
one of Z¹³ or Z¹⁴ represents a sulfur atom or an oxygen atom, and the other of Z¹³ or Z¹⁴ represents —CR═,
in Formula (A2), Z²¹ to Z²³ each independently represent a sulfur atom or an oxygen atom, and at least one of Z²¹, Z²², or Z²³ represents a sulfur atom,
in Formula (A3), Z³¹ and Z³² each independently represent a sulfur atom or an oxygen atom, and at least one of Z³¹ or Z³² represents a sulfur atom,
in Formula (A4), one of Z⁴¹ or Z⁴² represents a sulfur atom, and the other of Z⁴¹ or Z⁴² represents —CR═, and
one of Z⁴³ or Z⁴ represents a sulfur atom or an oxygen atom, and the other of Z⁴³ or Z⁴ represents —CR═,
in Formula (A5), one of Z⁵¹ or Z⁵² represents a sulfur atom, and the other of Z⁵¹ or Z⁵² represents —CR═, and
one of Z⁵³ or Z⁵⁴ represents a sulfur atom or an oxygen atom, and the other of Z⁵³ or Z⁵⁴ represents —CR═, and
in Formula (A6), Z⁶¹ to Z⁶⁴ each independently represent a sulfur atom or an oxygen atom, and at least one of Z⁶¹, Z⁶², Z⁶³, or Z⁶⁴ represents a sulfur atom.

4. The photoelectric conversion element according to claim 2,
wherein in Formula (1), n11 is 1, and Ar¹¹ is a group represented by Formula (A5).

5. The photoelectric conversion element according to claim 1,
wherein in Formula (2), X²¹ and X²² each independently represent a sulfur atom or an oxygen atom,
in Formula (3), X³¹ represents a sulfur atom or an oxygen atom, and
in Formula (4), X⁴¹ represents a sulfur atom or an oxygen atom.

6. The photoelectric conversion element according to claim 1,
wherein in Formula (1), Ar¹² and Ar¹³ each independently represent any one of groups represented by Formula (5) to Formula (13), 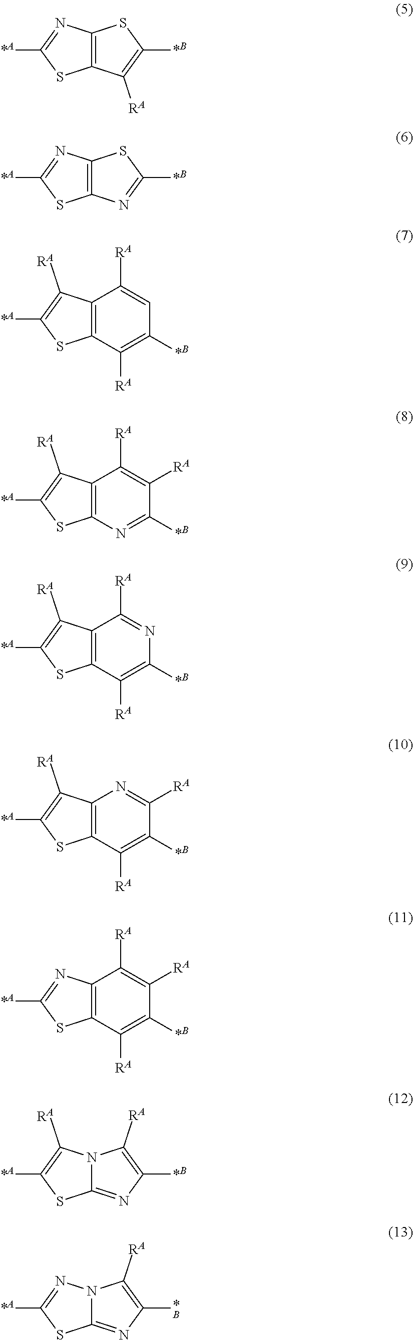
in Formula (5) to Formula (13), *A ad*B each represent a bonding position, and
R^{A} represents a hydrogen atom or a substituent.

7. The photoelectric conversion element according to claim 1,
wherein the compound represented by Formula (1) has a molecular weight of 550 to 1200.

8. The photoelectric conversion element according to claim 1,
wherein the photoelectric conversion film further contains a coloring agent, and
the photoelectric conversion film is a mixture layer formed in a state in which the compound represented by Formula (1) and the coloring agent are mixed.

9. The photoelectric conversion element according to claim 1,
wherein the photoelectric conversion film further contains a n-type semiconductor material.

10. The photoelectric conversion element according to claim 9,
wherein the n-type semiconductor material includes fullerenes selected from the group consisting of a fullerene and a derivative thereof.

11. The photoelectric conversion element according to claim 1, further comprising one or more interlayers between the conductive film and the transparent conductive film, in addition to the photoelectric conversion film.

12. An imaging element comprising the photoelectric conversion element according to claim 1.

13. An optical sensor comprising the photoelectric conversion element according to claim 1.

14. A compound represented by Formula (1), 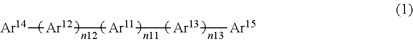
in Formula (1), n11 represents 1 or 2,
n12 and n13 each independently represent 0 or 1, where, at least one of n12 or n13 represents 1,
Ar¹¹ represents a fused polycyclic aromatic ring group consisting of a combination of one or more aromatic rings selected from the group consisting of a thiophene ring, a benzene ring, a furan ring, and a selenophene ring, where, the fused polycyclic aromatic ring group has three or four rings, the fused polycyclic aromatic ring group contains at least one thiophene ring, the fused polycyclic aromatic ring group may have a substituent,
Ar¹⁴ and Ar¹⁵ each independently represent an aryl group which may have a substituent or a heteroaryl group which may have a substituent, and
Ar¹² and Ar¹³ each independently represent any one of groups represented by Formula (2) to Formula (4), 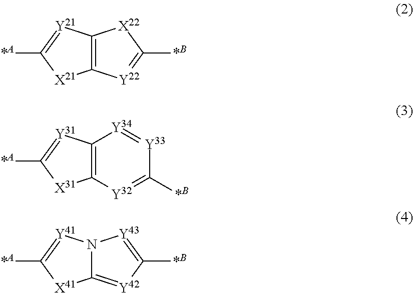
in Formula (2) to Formula (4), *^{A} and *^{B} each represent a bonding position,
in Formula (2), X²¹ and X²² each independently represent a sulfur atom, an oxygen atom, or a selenium atom, Y²¹ and Y²² each independently represent a nitrogen atom or —CR═, and R represents a hydrogen atom or a substituent, where, at least one of Y²¹ or Y²² represents a nitrogen atom,
in Formula (3), X³¹ represents a sulfur atom, an oxygen atom, or a selenium atom, Y³¹ to Y³⁴ each independently represent a nitrogen atom or —CR═, and R represents a hydrogen atom or a substituent,
in Formula (4), X⁴¹ represents a sulfur atom, an oxygen atom, or a selenium atom, Y⁴¹ to Y⁴³ each independently represent a nitrogen atom or —CR═, and R represents a hydrogen atom or a substituent.

15. The compound according to claim 14,
wherein in Formula (1), a group represented by Ar¹¹ is any one of groups represented by Formula (A1) to Formula (A6), 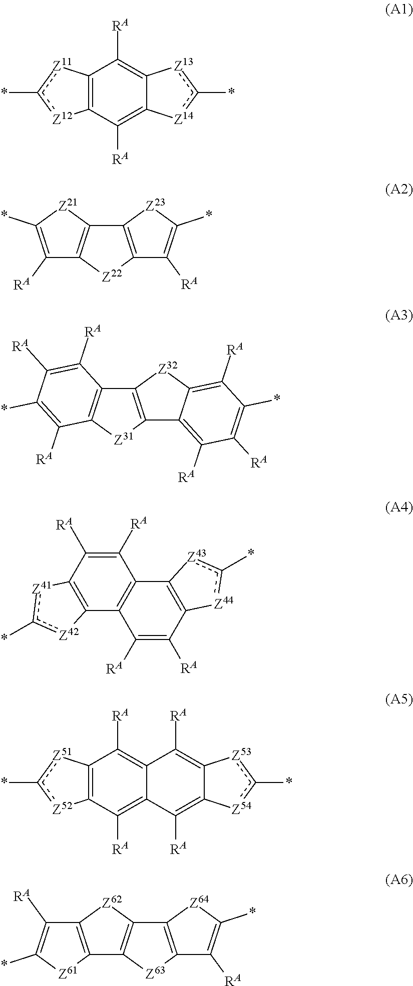
in Formulae (A1) to (A6), * represents a bonding position,
in Formula (A1), one of Z¹¹ or Z¹² represents a sulfur atom, and the other of Z¹¹ or Z¹² represents —CR═, one of Z¹³ or Z¹⁴ represents a sulfur atom, an oxygen atom, or a selenium atom, and the other of Z¹³ or Z¹⁴ represents —CR═, and R and R^{A} each independently represent a hydrogen atom or a substituent,
in Formula (A2), Z²¹ to Z²³ each independently represent a sulfur atom, an oxygen atom, or a selenium atom, at least one of Z²¹, Z²², or Z²³ represents a sulfur atom, and R^{A} represents a hydrogen atom or a substituent,
in Formula (A3), Z³¹ and Z³² each independently represent a sulfur atom, an oxygen atom, or a selenium atom, at least one of Z³¹ or Z³² represents a sulfur atom, and R^{A} represents a hydrogen atom or a substituent,
in Formula (A4), one of Z⁴¹ or Z⁴² represents a sulfur atom, and the other of Z⁴¹ or Z⁴² represents —CR═, one of Z⁴³ or Z⁴⁴ represents a sulfur atom, an oxygen atom, or a selenium atom, and the other of Z⁴³ or Z⁴⁴ represents —CR═, and R and R^{A} each independently represent a hydrogen atom or a substituent,
in Formula (A5), one of Z⁵¹ or Z⁵² represents a sulfur atom, and the other of Z⁵¹ or Z⁵² represents —CR═, one of Z⁵³ or Z⁵⁴ represents a sulfur atom, an oxygen atom, or a selenium atom, and the other of Z⁵³ or Z⁵⁴ represents —CR═, and R and R^{A} each independently represent a hydrogen atom or a substituent,
in Formula (A6), Z⁶¹ to Z⁶⁴ each independently represent a sulfur atom, an oxygen atom, or a selenium atom, at least one of Z⁶¹, Z⁶², Z⁶³, or Z⁶⁴ represents a sulfur atom, and R^{A} represents a hydrogen atom or a substituent.

16. The compound according to claim 15,
wherein in Formula (A1), one of Z¹¹ or Z¹² represents a sulfur atom, and the other of Z¹¹ or Z¹² represents —CR═, and
one of Z¹³ or Z¹⁴ represents a sulfur atom or an oxygen atom, and the other of Z¹³ or Z¹⁴ represents —CR═,
in Formula (A2), Z²¹ to Z²³ each independently represent a sulfur atom or an oxygen atom, and at least one of Z²¹, Z²², or Z²³ represents a sulfur atom,
in Formula (A3), Z³¹ and Z³² each independently represent a sulfur atom or an oxygen atom, and at least one of Z³¹ or Z³² represents a sulfur atom,
in Formula (A4), one of Z⁴¹ or Z⁴² represents a sulfur atom, and the other of Z⁴¹ or Z⁴² represents —CR═, and
one of Z⁴³ or Z⁴ represents a sulfur atom or an oxygen atom, and the other of Z⁴³ or Z⁴ represents —CR═,
in Formula (A5), one of Z⁵¹ or Z⁵² represents a sulfur atom, and the other of Z⁵¹ or Z⁵² represents —CR═, and
one of Z⁵³ or Z⁵⁴ represents a sulfur atom or an oxygen atom, and the other of Z⁵³ or Z⁵⁴ represents —CR═, and
in Formula (A6), Z⁶¹ to Z⁶⁴ each independently represent a sulfur atom or an oxygen atom, and at least one of Z⁶¹, Z⁶², Z⁶³, or Z⁶⁴ represents a sulfur atom.

17. The compound according to claim 15,
wherein in Formula (1), n11 is 1, and Ar¹¹ is a group represented by Formula (A5).

18. The compound according to claim 14,
wherein in Formula (2), X²¹ and X²² each independently represent a sulfur atom or an oxygen atom,
in Formula (3), X³¹ represents a sulfur atom or an oxygen atom, and
in Formula (4), X⁴¹ represents a sulfur atom or an oxygen atom.

19. The compound according to claim 14,
wherein in Formula (1), Ar¹² and Ar¹³ each independently represent any one of groups represented by Formula (5) to Formula (13), 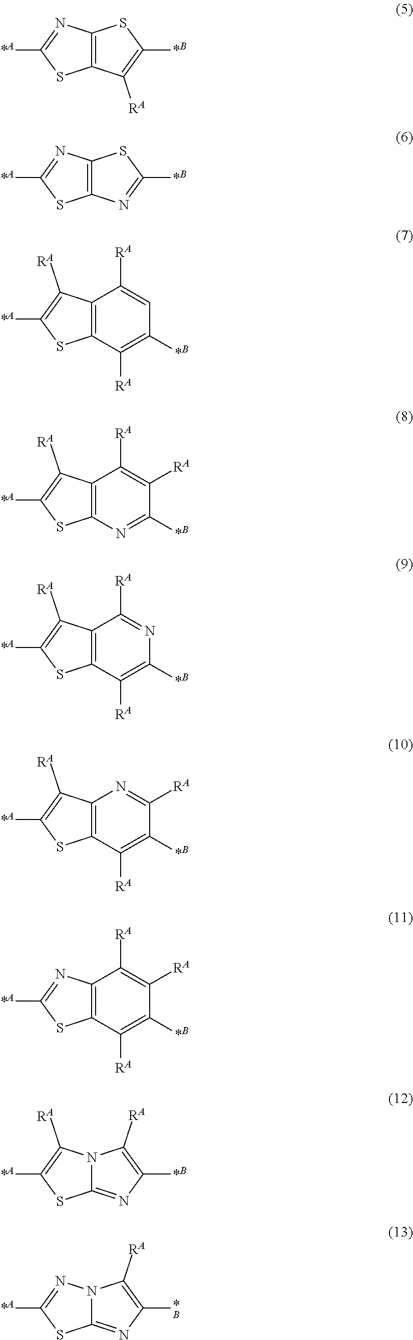
in Formula (5) to Formula (13), *^{A} and *^{B} each represent a bonding position, and
R^{A} represents a hydrogen atom or a substituent.

20. The compound according to claim 14,
wherein the compound represented by Formula (1) has a molecular weight of 550 to 1200.
